(19) United States
(12) Patent Application Publication
(10) Pub. No.: US 2024/0316012 A1
(43) Pub. Date: Sep. 26, 2024

(54) METHODS FOR TREATING LYMPHOMA

(71) Applicants: Northwestern University, Evanston, IL (US); Technische Universität München, München (DE)

(72) Inventors: Jaehyuk Choi, Chicago, IL (US); Jay Carroll Daniels, Chicago, IL (US); Jurgen Ruland, München (DE); Tim Wartewig, Higganum, CT (US)

(73) Assignees: Northwestern University, Evanston, IL (US); Technische Universität München, München (DE)

(21) Appl. No.: 18/614,107

(22) Filed: Mar. 22, 2024

Related U.S. Application Data
(60) Provisional application No. 63/454,187, filed on Mar. 23, 2023.

(51) Int. Cl. A61K 31/436; A61K 31/18; A61K 31/7004; A61P 35/00

(52) U.S. Cl. CPC A61K 31/436; A61K 31/18; A61K 31/7004; A61P 35/00

(57) ABSTRACT

Provided herein is a method for treating a lymphoma in a subject in need thereof, comprising administering to the subject an effective amount of at least one PD-1 pathway agonist.

### CROSS REFERENCE TO RELATED APPLICATIONS

This application claims the benefit under 35 U.S.C. § 119(e) of U.S. Provisional Application Ser. No. 63/454,187, filed Mar. 23, 2023, the contents of which are hereby incorporated by reference in its entirety.

### STATEMENT OF GOVERNMENT SUPPORT

This invention was made with government support under grant number 1DP2AI136599-01 awarded by the National Institutes of Health. The government has certain rights in the invention.

### TECHNICAL FIELD

The present technology relates generally to the treatment of cancer. More specifically, the present technology relates to the treatment of lymphoma.

### SEQUENCE LISTING

The instant application contains a Sequence Listing which has been submitted electronically in XML format and is hereby incorporated by reference in its entirety. Said XML copy, created on May 20, 2024, is named 121384-0226_SL.xml and is 23,010 bytes in size.

### BACKGROUND

The following description of the background of the present technology is provided simply as an aid in understanding the present technology and is not admitted to describe or constitute prior art to the present technology.

The PDCD1 encoded immune checkpoint receptor PD-1 is a key tumor suppressor in T cells that is recurrently inactivated in T cell non-Hodgkin lymphomas (T-NHLs). The highest frequencies of PDCD1 deletions are detected in advanced disease, predicting inferior prognosis. However, the tumor-suppressive mechanisms of PD-1 signaling remain unknown.

### SUMMARY OF THE PRESENT TECHNOLOGY

In one aspect, the present disclosure provides a method of treating lymphoma in a subject in need thereof. In some aspects, the method comprises, consists of, or consists essentially of administering to the subject an effective amount of at least one PD1 pathway agonist. In some aspects, the lymphoma is a T cell non-Hodgkin lymphoma.

In some aspects, the PD-1 pathway agonist is selected from the group comprising, consisting of, or consisting essentially of a PD-1 agonist, a PI3K-AKT-mTOR pathway inhibitor, a glycolysis inhibitor, an ACLY inhibitor, an AP1 inhibitor, or any combination of two or more thereof. In some aspects, the PD-1 agonist comprises, consists of or consists essentially of ImmTAA1 molecules, PD-L1, Rosnilimab, or any combination of two or more of.

In some aspects, the PI3K-AKT-mTOR pathway inhibitor is selected from the group comprising, consisting of, or consisting essentially of a PI3K inhibitor, an AKT inhibitor, an mTOR inhibitor, or any combination of two or more thereof. In some aspects, the PI3K inhibitor is selected from the group comprising, consisting of, or consisting essentially of alpelisib, AMG319, apitolisib, AZD8186, BKM120, BGT226, bimiralisib, buparlisib, CH5132799, copanlisib, CUDC-907, dactolisisb, duvelisib, GDC-0941, GDC-0084, gedatolisib, GSK2292767, GSK2636771, idelalisib, IPI-549, leniolisib, LY294002, LY3023414, nemiralisib, omipalisib, PF-04691502, pictilisib, pilaralisib, PX866, RV-1729, SAR260301, SAR245408, serabelisib, SF 1126, sonolisib, taselisib, umbralisib, voxtalisib, VS-5584, wortmannin, WX-037, ZSTK474, or any combination of two or more thereof.

In some aspects, the AKT inhibitor is selected from the group comprising, consisting of, or consisting essentially of MK-2206, A-674563, A-443654, acetoxy-tirucallic acid, 3α- and 3β-acetoxy-tirucallic acids, afuresertib (GSK2110183), 4-amino-pyrido[2,3-d]pyrimidine derivative API-1,3-aminopyrrolidine, anilinotriazole derivatives, ARQ751, ARQ 092, AT7867, AT13148, 7-azaindole, AZD5363, (−)-balanol derivatives, BAY 1125976, Boc-Phe-vinyl ketone, CCT128930, 3-chloroacetylindole, diethyl 6-methoxy-5,7-dihydroindolo [2,3-b]carbazole-2,10-dicarboxylate, diindolylmethane, 2,3-diphenylquinoxaline derivatives, DM-PIT-1, edelfosine, erucylphosphocholine, erufosine, frenolicin B, GSK-2141795, GSK690693, H-8, H-89, 4-hydroxynonenal, ilmofosine, imidazo-1,2-pyridine derivatives, indole-3-carbinol, ipatasertib, kalafungin, lactoquinomycin, medermycin, 3-methyl-xanthine, miltefosine, 1,6-naphthyridinone derivatives, NL-71-101, N-[(1-methyl-1H-pyrazol-4-yl)carbonyl]-N′-(3-bromophenyl)-thiourea, OSU-A9, perifosine, 3-oxo-tirucallic acid, PH-316, 3-phenyl-3H-imidazo[4,5-b]pyridine derivatives, 6-phenylpurine derivatives, PHT-427, PIT-1, PIT-2, 2-pyrimidyl-5-amidothiophene derivative, pyrrolo[2,3-d]pyrimidine derivatives, quinoline-4-carboxamide, 2-[4-(cyclohexa-1,3-dien-1-yl)-1H-pyrazol-3-yl]phenol, spiroindoline derivatives, triazolo[3,4-f][1,6]naphthyridin-3(2H)-one derivative, triciribine, triciribine mono-phosphate active analogue, uprosertib, or any combination of two or more thereof.

In some aspects, the mTOR inhibitor is selected from the group comprising, consisting of, or consisting essentially of Torin, CCI-779, AZD2014, AZD8055, CC-223, dactolisib, everolimus, GSK2126458, Ku-0063794, Ku-0068650, MLN0128, OSI027, PP242, RapaLinks, rapamycin, ridaforolimus, sapanisertib, temsirolimus, vistusertib, WAY-600, WYE-687, WYE-354, XL765, or any combination of two or more thereof. In some aspects, the Torin comprises, consists of, or consists essentially of Torin 1 and/or Torin 2

In some aspects, the glycolysis inhibitor is selected from the group comprising, consisting of, or consisting essentially of 2-deoxy-D-glucose, 3-bromopyruvic acid, 6-aminonicotinamide, lonidamine, oxythiamine chloride hydrochloride, or any combination of two or more thereof.

In some aspects, the ACLY inhibitor is selected from the group comprising, consisting of, or consisting essentially of BMS303141, SB204990, or any combination of two or more thereof.

In some aspects, the AP1 inhibitor is selected from the group comprising, consisting of, or consisting essentially of T-5224, SP100030, SPC-839, K1115A, Momordin I, or any combination of two or more thereof.

In yet another aspect, the method further comprises, consists of, or consists essentially of administering to the subject an additional therapeutic agent. In some aspects, the at least one PD-1 pathway agonist and the additional therapeutic agent are administered separately, sequentially, or simultaneously.

In some aspects, the lymphoma is resistant to radiation therapy, chemotherapy or immunotherapy. In yet another aspect, the subject is non-responsive to at least one prior line of cancer therapy. In some aspects, the at least one prior line of cancer therapy comprises, consists of, or consists essentially of radiation therapy, chemotherapy or immunotherapy.

In some aspects, the at least one PD-1 pathway agonist is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically.

In some aspects, the subject is a human subject. In some aspects, the subject exhibits decreased tumor growth, reduced tumor proliferation, lower tumor burden, or increased survival after administration of the at least one PD-1 pathway agonist.

### BRIEF DESCRIPTION OF THE DRAWINGS

FIGS. 1A-1I show that loss of Pdcd1 enables oncogene-enforced glycolysis in T cells. FIG. 1A schematically illustrates transgenic ITK-SYK allele with eGFP reporter sequence and cre-induced excision of the STOP cassette. TAM, tamoxifen. FIG. 1B shows the experimental strategy to explore early molecular events upon ITK-SYK induction in the presence or absence of Pdcd1. FIG. 1C shows the top 20 differentially expressed genes between ITK-SYKCD4-creERT2-(“Pdcd1+/+”) and ITK-SYKCD4-creERT2; Pdcd1−/−-(“Pdcd1−/−”) derived ITK-SYK expressing T cells. RNA-seq was performed using spleen-derived eGFP+ T cells, FACS-sorted on day 5 (d5) after Tamoxifen injection (TAM) (n=4 mice per group). Expression values are normalized z-scores. FIG. 1D illustrates GSEA of the indicated signatures. FDR=color intensity of circles; NES=circle diameter. Blue/red indicates the group in which the signature was positively enriched; NES, normalized enrichment score; FDR, false discovery rate. FIG. 1E shows flow cytometry for HIF1α in ITK-SYK-expressing T cells. Spleen-derived single-cell suspensions were generated from ITK-SYKCD4-creERT2 (“Pdcd1+/+”) and ITK-SYKCD4-creERT2; Pdcd1−/− (“Pdcd1−/−”) mice on d5 after TAM. FIG. 1F shows western blot analysis from lysates of ITK-SYK-expressing eGFP+ T cells, FACS-sorted from ITK-SYKCD4-creERT2 (“Pdcd1+/+”) and ITK-SYKCD4-creERT2; Pdcd1−/− (“Pdcd1−/−”) mice on d5 after TAM. FIG. 1G shows ECAR metabolic flux analysis of ITK-SYK-expressing CD4+ T cells isolated from ITK-SYKCD4-creERT2 (“Pdcd1+/+”) and ITK-SYKCD4-creERT2; Pdcd1−/− (“Pdcd1−/−”) mice d5 after TAM. (n=3 biological replicates per group). Data was normalized using total cellular protein. ECAR, extracellular acidification rate. P=two-sided Student's t-test. Middle line denotes the median, the top and bottom box edges denote the 0.25 and 0.75 quantiles, respectively, and the whiskers denote the minimum and maximum values. FIG. 1H shows OCR metabolic flux analysis from the same experiment as in (g). OCR, oxygen consumption rate. P=two-sided Student's t-test. FIG. 1I shows lactate concentration in cell culture supernatants. ITK-SYK-expressing eGFP+ cells were FACS-sorted from ITK-SYKCD4-creERT2 (“Pdcd1+/+”) and ITK-SYKCD4-creERT2; Pdcd1−/− (“Pdcd1−/−”) mice on d5 after TAM and incubated overnight in vitro. (n=3 and n=4 biological replicates per group). Data was normalized using viable cell numbers. P=two-sided Student's t-test. Shown are the mean±SD and individual data points. FIGS. 1C and 1D, Data from one experiment. FIGS. 1E and 1F, Representative data from two independent experiments with two biological replicates per group. FIGS. 1G and 1H Representative data from two independent experiments with three biological replicates per group. FIGS. 1I, Representative data from two independent experiments

FIGS. 2A-2F PD-1 represses mTOR and HIF1α in pre-malignant cells. FIG. 2A shows whole-body SUV maps from ¹⁸F-FDG-PET measurements of C57BL 6 mice, ITK-SYK^{CD4-creERT2} (“Pdcd1^{+/+}”) and ITK-SYK^{CD4-creERT2}; Pdcd1^{-/-} (“Pdcd1^{-/-}”) mice together with anatomical MRI acquisition. ¹⁸F-FDG-PET measurements were conducted on d5 after TAM application. ¹⁸F-FDG-PET signals from lymph nodes were magnified (insets). White arrows indicate spleens. Color intensity represents the calculated MBq/ml uptake of ¹⁸F-FDG. SUV, standardized uptake value ¹⁸F-FDG, ¹⁸F-fluordesoxyglucose. FIG. 2B shows SUVₘₐₓ for the indicated genotypes (n=3 for C57BL/6, n=9 for Pdcd1^{+/+}, and n=6 for Pdcd1^{-/-}) from the experiment shown in (a). SUVₘₐₓ denotes the maximum signal found in a single voxel across the entire tumor lesion. P=one-way ANOVA and two-sided Student's t-test. Shown are the mean±SEM and individual data points. FIG. 2C shows Exemplary 7T-MRI CSI derived ¹³C-MR spectra showing [1-¹³C]pyruvate and [1-¹³C]lactate amplitudes in the splenic ROI of ITK-SYK^{CD4-creERT2} (“Pdcd1^{+/+}”) and ITK-SYK^{CD4-creERT2}; Pdcd1^{-/-} (“Pdcd1^{-/-}”) mice following the injection of hyperpolarized [1-¹³C]pyruvate on d5 after TAM. CSI, chemical shift imaging. ROI, region of interest. FIG. 2D shows Ratio of [1-¹³C]pyruvate and [1-¹³C]lactate amplitudes in spleens measured by 7T-MIRI in ITK-SYK^{CD4-creERT2} (“Pdcd1^{+/+}”) and ITK-SYK^{CD4-creERT2}; Pdcd1^{-/-} (“Pdcd1^{-/-}”) mice on d5 after TAM (n=3 mice per genotype). P=two-sided Student's t-test. Shown are the mean±SD and individual data points. FIG. 2E shows flow cytometry for p-AKT^{S473}, p-mTOR^{S2448}, HIF1α, glut 1, and hexokinase 2 in ITK-SYK-expressing T cells. Spleen-derived single-cell suspensions were generated from ITK-SYK^{CD4-creERT2} mice on d5 after TAM. Before fixation, anti-PD-L1 or control antibodies were added for four hours in vitro. FIG. 2F shows survival of ITK-SYK⁺PD-1⁻ lymphoma-bearing NSG mice treated with Torin-1 or vehicle. On day 0, NSG mice received 1,000 ITK-SYK-expressing eGFP+ cells from an ITK-SYK^{CD4-creERT2}; Pdcd1^{-/-} mouse, FACS-sorted on d5 after TAM. Mice received 6 mg/kg Torin-1 or vehicle daily via intraperitoneal injection (n=6 mice per group) from day 3 p.i. on. P=two-sided log-rank test. FIG. 2A, Representative mice from two independent experiments. FIG. 2B, Pooled data from two independent experiments. FIG. 2C, Representative amplitudes from one experiment. FIG. 2D, Pooled data from two independent experiments. FIG. 2E, Representative data from two independent experiments with two biological replicates per group. FIG. 2F, Pooled data from two independent experiments

FIGS. 3A-3I shows that PD-1 facilitates glycolysis-dependent histone-acetylation. FIG. 3A shows intracellular ¹³C abundance within the glycolysis pathways detected by targeted LC-MS/MS relative to intracellular [U-¹³C]glucose in ITK-SYK⁺ T cells+/−PD-1, cultured with uniformly labeled [U-¹³C]glucose for 3 h in vitro. Data are normalized to viable cell number and z-scores per metabolite. FIG. 3B shows Time-dependent ATP measurement in ITK-SYK⁺ T cells+/−PD-1. P=two-sided Student's t-test. The data are presented as the mean±SEM and individual data points for each animal. Cross indicates death of the animal. FIG. 3C shows ion count for intracellular ¹³C labeled metabolites in the pentose phosphate pathway. FIG. 3D shows ion count for intracellular ¹³C-citrate (m+2) isotopomers. P=two-sided Student's t-test. Shown are the mean±SD and individual data points. FIG. 3E shows western blot analysis of acetylated histones in ITK-SYK⁺ T cells+/−PD-1. FIG. 3F shows flow cytometry for H3K27ac in wild-type CD4+ or ITK-SYK⁺ T cells after a 4 h in vitro incubation with different glucose concentrations. r=Pearson's correlation coefficient. Data are presented as mean±SD and individual data points. FIG. 3G shows flow cytometry for H3K27ac in ITK-SYK⁺PD-1⁻ cells after 4 h in vitro culture with 2-DG (1 mM) or DMSO. P=paired two-sided Student's t-test. Data are presented as the mean±SD. FIG. 3H shows [U-¹³C] glucose-derived intracellular [1,2-¹³C]acetyl-CoA levels determined by targeted LC-MS/MS in ITK-SYK⁺ T cells+/−PD-1 cultured with uniformly labeled [U-¹³C]glucose (11 mM) for 4 h in vitro (n=3 mice genotype). Data was normalized as in (a). P=two-sided Student's t-test. The data are presented as the mean±SEM. FIG. 3I shows [U-¹³C] glucose-derived ¹³C-H3K27ac (m+2) determined by orbitrap LC-MS/MS. Experiment as in (h) (n=3 mice genotype). Data was normalized as in (a). P=two-sided Student's t-test. Data are presented as the mean±SD. FIGS. 3-D, Representative data from two independent experiments with n=3 biological replicates per group. FIG. 3E, Representative data from two independent experiments with two biological replicates per group. FIG. 3F, Representative data from two independent experiments. FIG. 3G, Representative data from two independent experiments with three biological replicates per group. FIG. 3H, Representative data from one experiment with four biological replicates per group. FIG. 3I, Representative data from one experiment with three biological replicates per group

FIGS. 4A-4H show ACLY is a critical effector molecule downstream of PD-1. FIG. 4A shows a schematic of glucose flux to histone acetyl groups. FIG. 4B shows flow cytometry for H3K27ac in ITK-SYK⁺ cells after 4 h in vitro culture with the ACLY inhibitor BMS-303141 (“iACLY,” 15 μM) or DMSO. ITK-SYK⁺ cells were FACS-sorted from ITK-SYK^{CD4-creERT2} mice on d5 after TAM and 12 h anti-PD-L1 or isotype control antibody treatment (i.p., 200 μg). P=paired two-sided Student's t-test. Data are presented as the mean±SD. FIG. 4C shows western blot analysis in lysates from ITK-SYK⁺PD-1⁺ cells. Spleen-derived single-cell suspensions were FACS-sorted from ITK-SYK^{CD4-creERT2} mice on d5 after TAM and cultured in vitro overnight with anti-PD-L1 or isotype control antibodies and the PI3K inhibitors (“iPI3K”) Wortmannin (20 nM), LY294002 (20 μM), or DMSO. FIG. 4D shows flow cytometry for CD4 and eGFP in viable lymphocytes derived from spleens of acutely induced ITK-SYK^{CD4-creERT2}; Pdcd1^{-/-} mice after three day in vitro culture with BMS-303141 (“iACLY,” 5 μM) or DMSO. FIG. 4E shows Fold change in viable ITK-SYK expressing eGFP+ cells in media supplemented with the BMS-303141 (“iACLY,” 5 μM) or DMSO. Spleen-derived lymphocytes, FACS-sorted from ITK-SYK^{CD4-creERT2} and ITK-SYK^{CD4-creERT2}; Pdcd1^{-/-} mice on d5 after TAM were incubated for three days in vitro. P=two-sided Student's t-test. Data are presented as the mean±SD. FIG. 4F shows western blot for ACLY in ITK-SYK⁺PD-1⁻ cells electroporated with Acly-targeting (sgACLY) or non-targeting control (MOCK) RNP complexes. Protein lysates were generated after 48 h in vitro culture. RNP, ribonucleoprotein. FIG. 4G shows survival of recipient mice transplanted with Acly (“sgACLY”) or MOCK-edited (“sgMOCK”) ITK-SYK⁺PD-1⁻ cells. P=two-sided Logrank-test. FIG. 4H shows gene-wise global H3K27ac scores from anti-H3K27ac ChIP-seq in a 1.5 Kb window around TSS. ITK-SYK⁺ cells were FACS-sorted from ITK-SYK^{CD4-creERT2} (“Pdcd1^{+/+}”) and ITK-SYK^{CD4-creERT2}; Pdcd1^{-/-} (“Pdcd1^{-/-}”) mice on d5 after TAM, fixed, and processed for anti-H3K27ac ChIP-seq analysis. ChIP-seq, chromatin immunoprecipitation sequencing; TSS, transcription start site. FIG. 4B, Representative data from three independent experiments with three biological replicates per group. FIG. 4C, Representative data from two independent experiments with two biological replicates. FIGS. 4D and 4E, Representative data from 3 independent experiments. FIGS. 4F and 4G, Data from one experiment with n=5 mice per group. FIG. 4H, Data from a single experiment with two biological replicates per group

FIGS. 5A-5H show PD-1 controls AP-1 activity in an acetyl-CoA-dependent manner. FIG. 5A shows Differential transcription factor (TF) footprint analysis of ATAC-seq data. ITK-SYK⁺ cells were FACS-sorted from ITK-SYK^{CD4-creERT2} (“Pdcd1^{+/+}”) and ITK-SYK^{CD4-creERT2}; Pdcd1^{-/-} (“Pdcd1^{-/-}”) mice on d5 after TAM and immediately processed for ATAC-seq. JASPAR database motif names for the selected AP-1 family TFs are indicated. Enrichment of a TF motif is indicated by motif score, motif rank, and color. FIG. 5B shows Binding profiles of selected TFs for indicated genotypes from the experiment shown in (a). FIG. 5C shows ChIP-enrich analysis of ChIP-seq data shown in (FIG. 4h) for the indicated gene sets. FDR=color intensity of the circles. ChIP-seq peaks in gene set=circle diameter. Blue/red indicates the genotype in which a signature was positively enriched. FDR, false discovery rate. FIG. 5D shows GSEA for the indicated gene sets. FDR=color intensity of circles. NES=circle diameter. Blue/red indicates the group in which a signature was positively enriched. NES, normalized enrichment score; FDR, false discovery rate. FIG. 5E shows Phosflow analysis of p-c-Fos^{S32} in ITK-SYK⁺ cells. Single-cell suspensions were generated from lymph nodes of ITK-SYK^{CD4-creERT2} mice on d5 after TAM and 12 h anti-PD-L1 or isotype control antibody treatment (i.p., 200 g). P=paired two-sided Student's t-test. Data are presented as the mean±SD. FIG. 5F shows Phosflow analysis of p-c-Jun^{S73}. Same experiment as in (e). FIGS. 5G and 5H show Differential TF footprint analysis of ATAC-seq data from ITK-SYK⁺PD-1⁺ and ITK-SYK⁺PD-1⁻ cells after 3 hour in vitro incubation with BMS-303141 (“iACLY,” 5 μM, h) or DMSO (g). Spleen-derived eGFP+ cells were FACS-sorted from ITK-SYK^{CD4-creERT2} (“Pdcd1^{+/+}”) and ITK-SYK^{CD4-creERT2}; Pdcd1^{-/-} (“Pdcd1^{-/-}”) mice on d5 after TAM, incubated in iACLY or DMSO containing media and processed for ATAC-seq. Legend as in (a). FIGS. 5A and 5B: Data from a single experiment with three biological replicates per genotype. FIG. 5C, Data from one experiment with two biological replicates per group. FIG. 5D, Data from a single experiment with four biological replicates per genotype. FIGS. 5E and 5F, Representative data from 2 independent experiments with three biological replicates per group. FIGS. 5G and 511, Data from one experiment with three biological replicates per genotype.

FIGS. 6A-6E show PD-1 inactivation and glycolytic switch in hyperprogressive T-NHLs. FIG. 6A shows serial longitudinal analysis of the tumor burden index and WBC count for three individual CTCL patients with hyperprogressive disease. WBC, white blood cell count. Hyperprogression, rapid increase in tumor burden index (“post,” shaded in red) after an extended period of stable disease course (“pre,” shaded in blue). FIG. 6B shows differential gene expression analysis of RNA-seq data derived from the three CTCL patients in (a) before (“pre”) and after hyperprogression (“post”). Data points for selected glycolysis-related genes and PDCD1 are indicated. log₂FC, log₂(Fold Change). P=two-sided Wald-test. FIG. 6C shows RNA-seq read count of PDCD1 transcript before (“pre”) and after hyperprogression (“post”) shown for each patient individually. FIG. 6D shows WGS-based copy number aberration analysis of PDCD1-containing chromosomal region 2q36.3 to 2q37.3. The time point of genomic DNA isolation relative to hyperprogression is indicated (“pre” vs. “post”). The patient shown corresponds to the first patient in panel (a). Top, the dashed box indicates the genomic region on human chromosome 2 that is shown in detail at the bottom of the panel. Bottom, copy number aberration analysis. The location of the PDCD1 locus is shown. WGS, whole-genome sequencing. FIG. 6E: Gene set enrichment analyses for the indicated gene sets based on RNA-seq data derived from three patients with hyperprogression (a). The timepoint of RNA extraction relative to hyperprogression is indicated (“pre” vs. “post”). FDR=color intensity of circles NES=circle diameter. Blue/red indicates the group in which a signature was positively enriched; NES, normalized enrichment score; FDR, false discovery rate. FIG. 6A, Longitudinally collected data from three individual patients. FIGS. 6B-6E, Data from a single experiment with three patients.

FIGS. 7A-7H show ACLY dependency and AP-1 activation in human PDCD1 defective T-NHL. FIG. 7A shows Genomic region within q37.3 on chromosome 2 in 21 patients with CTCL. CNA, copy number aberrations. FIG. 7B GSEA for the indicated gene sets based on RNA-seq data from the 21 CTCL patients shown in (a). NES, normalized enrichment score; FDR, false discovery rate. FIG. 7C shows Phosflow analysis of serine 240/244 of S6 ribosomal protein in PDCD1mut (n=3), PDCD1 wt (n=3) primary CTCL cells and in CD4+ T cells from healthy donors (n=3). P=one-way ANOVA and two-sided Student's t-test. Shown are the mean±SD and individual data points. FIG. 7D shows uptake of the glucose analogue 2-NBDG in PDCD1mut (n=3), PDCD1 wt (n=3) primary CTCL cells and in CD4+ T cells from healthy donors (n=3). FACS-sorted malignant cells were stimulated with anti-CD3/anti-CD28 beads, and 2-NBDG uptake was determined at the indicated time points. P=two-sided Student's t-test. Shown are the mean±SEM and individual data points. FIG. 7E shows elative division index of malignant T cells from three patients with mutated PDCD1 (“PDCD1mut”) or reduced PD-1 expression after hyperprogression (“post”) compared to three “PDCD1 wt” and healthy CD4+ T cells (n=3) in the presence of 2-DG (1 mM), everolimus (0.1 μM), or BMS-303141 (iACLY, 10 μM). CFSE, carboxyfluorescein succinimidyl ester. P=two-sided Student's t-test. The data are presented as the mean±SEM and individual data points. FIG. 7F shows differential transcription factor (TF) footprint analysis of ATAC-seq data from three CTCL patients with (“PDCD1mut”) and three patients without (“PDCD1 wt”) mutated PDCD1. Legend as in (5a). FIG. 7G shows inding profile for the JASPAR FOS::JUN heterodimer motif for indicated genotypes from the experiment shown in (f). FIG. 7H GSEA for the indicated gene sets with RNA-seq data from 21 CTCL PDCD1mut” or “PDCD1 wt, and three patients with hyperprogression. NES, normalized enrichment score; FDR, false discovery rate. FIGS. 7A and 7B, Data from a single experiment with 21 patients. FIGS. 7C and 7D, Data from one experiment with three patients per group and three healthy donors. FIG. 7E, Data from one experiment with three patients and three healthy donors. FIGS. 7F and 7G, Data from one experiment with three patients per group. FIG. 7H, Data from a single experiment.

FIGS. 8A and 8B. FIG. 8A shows loss of tumor suppressor gene Pdcd1 is sufficient to enable immediate unrestricted clonal expansion of lymphomatous T cells upon single oncogene expression that is lethal to the host. FIG. 8B shows enhanced glucose uptake in Pdcd1-deficient T cells compared to the wild-type cells.

FIGS. 9A-9F. FIG. 9A show maximum 18F-FDG uptake in individual mice was measured in the spleens of both ITK-SYKCD4-creERT2 and ITK-SYKCD4-creERT2; Pdcd1−/− mice. FIG. 9B shows imaging demonstrating enhanced lactate production in vivo. FIG. 9C shows the injection of anti-PD-L1 into tamoxifen-treated ITK-SYK^{CD4-crERT} mice induced an unrestricted expansion of oncogene-expressing T cells, which rapidly killed the host. FIGS. 9D-F show acutely PD-1 inhibited cells also switched to glycolysis with an increase in ECAR and no change in their basal OCR.

FIG. 10A-10H. FIGS. 10A-10C show direct inhibition of glycolysis with 2-deoxy-D-glucose (2-DG) reduced the production of lactate, similar results were observed with the inhibition of mTOR or HIF1α. FIGS. 10D-10F show all three treatments of 2-DG and inhibition of mTOR or HIF1a were toxic to PD-1 deficient lymphoma cells. FIG. 10G shows treatment of ITK-SYK⁺PD-1⁻ lymphoma-bearing mice with the mTOR inhibitor Torin-1, which blocks mTOR activity within the tumor cells in vivo. FIG. 10H shows treatment of ITK-SYK⁺PD-1⁻ lymphoma-bearing mice with the mTOR inhibitor Torin-1 significantly prolonged survival.

FIG. 11A-11K. FIG. 11A shows both the genetic deletion of Pdcd1 and acute pharmacological blockade of PD-1 triggered a significant increase in histone H4 and H3 lysine 27 (H3K27) acetylation upon oncogenic T cell signaling. FIG. 11B shows the levels of [1,2-¹²C]-acetyl-CoA did not differ in transformed ITK-SYK-PD-1⁻ T cells from directly incubated ITK-SYK-expressing T cells from tamoxifen-injected ITK-SYK^{CD4-crERT2} and ITK-SYK^{CD4-creERT2}; Pdcd1^{-/-} mice ex vivo. FIG. 11C shows that in contrast, there was a significant increase in [1,2-¹³C]-acetyl-CoA and total acetyl-CoA ([1,2-¹²C]-acetyl-CoA+[1,2-¹³C]-acetyl-CoA) generation in transformed ITK-SYK⁺PD-1⁻ T cells. FIG. 11D shows increased de novo acetylation of histones H3 and H4 with [U-¹³C]glucose-derived ¹³C-acetyl-marks at H3K27, H3K9, H3K14, and H3K18/K23 in the transformed ITK-SYK⁺PD-1⁻ T cells compared to PD-1 proficient ITK-SYK⁺PD-1⁻ cells. FIG. 11E shows exogenous supplementation of acetate at physiological levels (100 μM) was not able to restore decreased H3K27ac signals in ACLY inhibitor-treated lymphoma cells. FIG. 11F shows treatment killed the ITK-SYK⁺PD-1⁻ lymphoma cells transformed ITK-SYK⁺PD-1⁻ T cells from tamoxifen-injected ITK-SYK^{CD4-creERT2}; Pdcd1^{-/-} mice with an ACLY inhibitor. FIGS. 11G and 11I shows the control-edited ITK-SYK⁺PD-1⁻ cells proliferated rapidly in vivo, as measured by eGFP monitoring, and killed all recipient animals as expected in less than 80 days, the ACLY-deficient ITK-SYK⁺PD-1⁻ T cells were unable to expand. In contrast, inhibition of the fatty acid synthase did not reveal a significant difference (FIGS. 11H and 11I). FIGS. 11J and 11K show the deletion of Pdcd1 increased H3K27 acetylation in oncogene-expressing T cells, particularly within promoter regions and around gene transcription start sites (TSS), indicating direct effects on transcriptional regulation.

FIGS. 12A and 12B. FIG. 12A shows a schematic model demonstrating a link between glucose-derived acetyl-CoA availability and selective opening of compact chromatin at AP-1 binding sites, which is mediated by the PD-1 pathway. FIG. 12B shows TCR clonotyping confirmed that post-hyperprogression samples originated from identical T cell clones as the pre-hyperprogression samples. Figure discloses SEQ ID NOS 2-25, respectively, in order of appearance.

FIG. 13A-13C. FIG. 13A shows PDCD1-mutant human tumors harbored diverse and numerous oncogenic TCR signaling mutations including in CD28, PLCG1, and RHOA, suggesting the transcriptional effects are not tied to the identity of the oncogene, and multiple PDCD1-mutant lymphomas carried mutations in another tumor suppressor, CDKN2A. FIGS. 13B and 13C the top transcription factor motifs most enriched in the PDCD1-mutant compared to the wild-type were AP-1 family members, including c-FOS, FOSL1, FOSL2, c-JUN, JUNB, and BATF, which we corroborated by functional in vitro assays.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999).

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Therapeutic Methods

In one aspect, the present disclosure provides a method of treating lymphoma in a subject in need thereof. In some aspects, the method comprises, consists of, or consists essentially of administering to the subject an effective amount of at least one PD-1 pathway agonist. In some aspects, the lymphoma is a T cell non-Hodgkin lymphoma.

In some aspects, the PD-1 pathway agonist is selected from the group comprising, consisting of, or consisting essentially of a PD-1 agonist, a PI3K-AKT-mTOR pathway inhibitor, a glycolysis inhibitor, an ACLY inhibitor, an AP1 inhibitor, or any combination of two or more thereof. In some aspects, the PD-1 agonist comprises, consists of or consists essentially of ImmTAA1 molecules, PD-L1, Rosnilimab, or any combination of two or more of.

In some aspects, the PI3K-AKT-mTOR pathway inhibitor is selected from the group comprising, consisting of, or consisting essentially of a PI3K inhibitor, an AKT inhibitor, an mTOR inhibitor, or any combination of two or more thereof. In some aspects, the PI3K inhibitor is selected from the group comprising, consisting of, or consisting essentially of alpelisib, AMG319, apitolisib, AZD8186, BKM120, BGT226, bimiralisib, buparlisib, CH5132799, copanlisib, CUDC-907, dactolisisb, duvelisib, GDC-0941, GDC-0084, gedatolisib, GSK2292767, GSK2636771, idelalisib, IPI-549, leniolisib, LY294002, LY3023414, nemiralisib, omipalisib, PF-04691502, pictilisib, pilaralisib, PX866, RV-1729, SAR260301, SAR245408, serabelisib, SF 1126, sonolisib, taselisib, umbralisib, voxtalisib, VS-5584, wortmannin, WX-037, ZSTK474, or any combination of two or more thereof.

In some aspects, the AKT inhibitor is selected from the group comprising, consisting of, or consisting essentially of MK-2206, A-674563, A-443654, acetoxy-tirucallic acid, 3α- and 3β-acetoxy-tirucallic acids, afuresertib (GSK2110183), 4-amino-pyrido[2,3-d]pyrimidine derivative API-1, 3-aminopyrrolidine, anilinotriazole derivatives, ARQ751, ARQ 092, AT7867, AT13148, 7-azaindole, AZD5363, (−)-balanol derivatives, BAY 1125976, Boc-Phe-vinyl ketone, CCT128930, 3-chloroacetylindole, diethyl 6-methoxy-5,7-dihydroindolo [2,3-b]carbazole-2,10-dicarboxylate, diindolylmethane, 2,3-diphenylquinoxaline derivatives, DM-PIT-1, edelfosine, erucylphosphocholine, erufosine, frenolicin B, GSK-2141795, GSK690693, H-8, H-89, 4-hydroxynonenal, ilmofosine, imidazo-1,2-pyridine derivatives, indole-3-carbinol, ipatasertib, kalafungin, lactoquinomycin, medermycin, 3-methyl-xanthine, miltefosine, 1,6-naphthyridinone derivatives, NL-71-101, N-[(1-methyl-1H-pyrazol-4-yl)carbonyl]-N′-(3-bromophenyl)-thiourea, OSU-A9, perifosine, 3-oxo-tirucallic acid, PH-316, 3-phenyl-3H-imidazo[4,5-b]pyridine derivatives, 6-phenylpurine derivatives, PHT-427, PIT-1, PIT-2, 2-pyrimidyl-5-amidothiophene derivative, pyrrolo[2,3-d]pyrimidine derivatives, quinoline-4-carboxamide, 2-[4-(cyclohexa-1,3-dien-1-yl)-1H-pyrazol-3-yl]phenol, spiroindoline derivatives, triazolo[3,4-f][1,6]naphthyridin-3(2H)-one derivative, triciribine, triciribine mono-phosphate active analogue, uprosertib, or any combination of two or more thereof.

In some aspects, the mTOR inhibitor is selected from the group comprising, consisting of, or consisting essentially of Torin, CCI-779, AZD2014, AZD8055, CC-223, dactolisib, everolimus, GSK2126458, Ku-0063794, Ku-0068650, MLN0128, OSI027, PP242, RapaLinks, rapamycin, ridaforolimus, sapanisertib, temsirolimus, vistusertib, WAY-600, WYE-687, WYE-354, XL765, or any combination of two or more thereof. In some aspects, the Torin comprises, consists of, or consists essentially of Torin 1 and/or Torin 2

In some aspects, the glycolysis inhibitor is selected from the group comprising, consisting of, or consisting essentially of 2-deoxy-D-glucose, 3-bromopyruvic acid, 6-aminonicotinamide, lonidamine, oxythiamine chloride hydrochloride, or any combination of two or more thereof.

In some aspects, the ACLY inhibitor is selected from the group comprising, consisting of, or consisting essentially of BMS303141, SB204990, or any combination of two or more thereof.

In some aspects, the AP1 inhibitor is selected from the group comprising, consisting of, or consisting essentially of T-5224, SP100030, SPC-839, K1115A, Momordin I, or any combination of two or more thereof.

In yet another aspect, the method further comprises, consists of, or consists essentially of administering to the subject an additional therapeutic agent. In some aspects, the at least one PD-1 pathway agonist and the additional therapeutic agent are administered separately, sequentially, or simultaneously.

In some aspects, the lymphoma is resistant to radiation therapy, chemotherapy or immunotherapy. In yet another aspect, the subject is non-responsive to at least one prior line of cancer therapy. In some aspects, the at least one prior line of cancer therapy comprises, consists of, or consists essentially of radiation therapy, chemotherapy or immunotherapy.

In some aspects, the at least one PD-1 pathway agonist is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically.

In some aspects, the subject is a human subject. In some aspects, the subject exhibits decreased tumor growth, reduced tumor proliferation, lower tumor burden, or increased survival after administration of the at least one PD-1 pathway agonist.

### EXAMPLES

The present technology is further illustrated by the following Examples, which should not be construed as limiting in any way.

### Example 1: Materials and Methods

Mice. Mice of both sexes aged 6-12 weeks were used for all experiments. Littermate controls were used whenever possible. Randomization and blinding were not performed. ITK-SYK^{CD4-creERT2} and ITK-SYK^{CD4-creERT2}; Pdcd1^{-/-} animals were described earlier and maintained on a C57BL 6 genetic background¹². Pdcd1^{-/-} (028276), B6J.129(Cg)-Gt(ROSA)26Sortm1.1(CAG-cas9*, -EGFP)Fezh/J (Cas9, 026179), and NOD.CG-Prkdcscid IL2rgtmlWjl/SzJ (NSG, 005557) mice were purchased from the Jackson Laboratory. All animal experiments were performed in accordance with local guidelines (Regierung von Oberbayern, Munich, Germany). Mice were euthanized if they exhibited signs of lymphoma (lymph node enlargement, palpable tumor, labored breathing, ascites) or if they lost 20% or more of their body weight. None of the approved thresholds were exceeded at any time. The mice were kept in greenline cages (Tecniplast Typ I superlong) at temperatures between 20-24° C. and 45-60% humidity, receiving pelleted food (No. 1324SP, Altromin) and autoclaved water ad libitum. A light-dark rhythm is changing every 12 hours with included dimming phases. For peripheral T-cell lymphomas both sexes showing nearly identical trends in survival, although incidence rates are higher in males compared to females (seer.cancer.gov). In accordance with this, Applicants used both female and male mice. Applicants indicate also that Applicants' mouse models did not reveal any differences in PD-1 expression and survival after PD-1 inactivation based upon sex. Applicants used the same numbers of male and female mice in each experiment whenever possible.

Induction of spontaneous ITK-SYK expression in peripheral T cells. Tamoxifen (T5648, Sigma) was dissolved in pure ethanol (with heating) and subsequently diluted in migliol (3274, Caesar & Loretz) to a final concentration of 10 mg/ml. To induce the expression of ITK-SYK in peripheral CD4⁺ T cells, ITK-SYK^{CD4-creERT2} and ITK-SYK^{CD4-ereERT2}; Pdcd1^{-/-} mice were intraperitoneally injected with tamoxifen. Therefore, Tamoxifen (T5648, Sigma) was dissolved in pure ethanol (with heating) before dilution in Peanut Oil (P21144, Sigma) to a final concentration of 10 mg/ml.

RNA-seq mouse. ITK-SYK^{CD4-creERT2} or ITK-SYK^{CD4-creERT2}; Pdcd1^{-/-} mice received a single dose of 2 mg tamoxifen. Five days later, the spleens were harvested and 1,000 eGFP+ ITK-SYK-expressing CD4⁺ T cells from both genotypes were directly sorted (FACSAria Fusion, BD Biosciences) into a 96-well PCR plate pre-filled with 10 μL of 1×TCL buffer (1070498, Qiagen) containing 1% (v/v) β-mercaptoethanol (M6250, Sigma). Library preparation for bulk 3-sequencing of poly(A)-RNA was performed as outlined earlier⁶⁰. Briefly, for each sample, barcoded full-length cDNA was generated with a Maxima RT polymerase (EP0742, Thermo) using oligo-dT primer-containing barcodes, unique molecular identifiers (UMIs), and an adapter. The addition of a template switch oligo (TSO) resulted in the extension of the 5 ends of the cDNAs, and full-length cDNA was amplified with a primer binding to the TSO site and the adapter. cDNA was fragmented using the Nextera XT kit (FC-131-1096, Illumina), and only the 3-end fragments were amplified using primers with Illumina P5 and P7 overhangs. Compared to⁶⁰, the P5 and P7 sites were exchanged to allow sequencing of the cDNA in read1 and barcodes and UMIs in read2 to achieve better cluster recognition. The library was sequenced on a NextSeq 500 platform (Illumina) with 75 cycles for the cDNA and 16 cycles for the barcodes and UMIs. Raw sequencing data was processed with DropSeq-tools (v1.12) using gene annotations from the Ensembl GRCm38.87 database to generate sample- and gene-wise UMI tables⁶¹. Downstream analysis was conducted using R v3.4.452 and DESeq2 v1.18.153⁶². Technical replicates with <100,000 total UMIs were excluded prior to differential expression analysis, and the remaining replicates were collapsed. Genes with <10 reads across all the conditions were excluded. Prior differential expression analysis and dispersion of the data were estimated using a parametric fit. GSEA 4.1.0 was used to calculate enrichment for the indicated signatures. All signatures were derived from MSigDB and Harmonizome^{51,63-65}.

Western blot analysis of cytosolic proteins and histones. ITK-SYK^{CD4-creERT2}, ITK-SYK^{CD4-creERT2}, Pdcd1^{-/-}, or C57BL 6 mice were intraperitoneally injected with a single dose of 2 mg tamoxifen and received 200 μg of anti-PD-L1 (BE0101, Bioxcell) or isotype control antibody (BE0090, Bioxcell), depending on the experimental condition. At the indicated time points, single cell suspensions were generated, eGFP⁺ cells were sorted by FACS and lysed on ice in protein lysis buffer (50 mM Tris, 150 mM NaCl, pH=8, 1% NP-40) supplemented with protease and phosphatase inhibitors (4906845001 and 539131, Sigma). The following antibodies were used in 1:1000 or 1:10000 (Actin) dilutions for protein detection: Hexokinase 2 (ab209847, Abcam), Phosphofructokinase-1/PFKM (MAB7687-SP, R&D Systems), Aldolase A (8060, CST), Enolase 1 (3810T, CST), Actin (3700, CST), Histone H3 (9715, CST), acetyl-Histone H3 Lys27 (8173, CST), Histone H4 (2935, CST), acetyl-Histone H4 (06-598, Millipore), ACLY (4332, CST), p-ACLY (4331, CST), AKT (4691, CST), and p-AKT (4060, CST). Analysis of p-ACLY^{S455}: For analysis of PD-1 dependent ACLY phosphorylation at serine 455, single cell suspensions from the spleens of acutely induced ITK-SYK^{CD4-creERT2} mice were incubated overnight with anti-PD-L1 antibody (4 μg/ml, BE0101, Bioxcell), isotype control (4 μg/ml, BE0090, Bioxcell), DMSO, Wortmannin (2 μM, 12-338, Sigma), or LY294002 (10 μM, 51105, Selleckchem). The next day, the cells were FACS-sorted for eGFP⁺ and lysed as described above. Analysis of histones: For western blotting of histones, single cell suspensions were generated from the spleens of acutely induced ITK-SYK^{CD4-creERT2} and ITK-SYK^{CD4-creERT2}; Pdcd1^{-/-} mice. Subsequently, FACS-sorted eGFP⁺ cells were cultured for four hours in in vitro in glucose-free Dulbecco's modified Eagle medium (DMEM; 11966025, Thermo) supplemented with 1% dialyzed fetal calf serum (FCS; A3382001, Thermo) and 1 mM glucose (G8270, Sigma). Next, cells were washed with ice-cold phosphate-buffered saline (PBS), and acid-based histone extraction was performed according to published protocols⁶⁶.

Glucose assay of cell culture supernatants. ITK-SYK^{CD4-creERT2} and ITK-SYK^{CD4-creERT2}; Pdcd1^{-/-} mice were intraperitoneally injected with a single dose of 2 mg tamoxifen. On day five, single cell suspensions were generated and eGFP⁺ cells were sorted by FACS. Two hundred thousand T cells were cultured in vitro in glucose-free DMEM (11966025, Thermo) supplemented with 10% dialyzed FCS (A3382001, Thermo), 2 mM glutamine (25030149, Thermo) and 10 mM glucose for 3 h. The remaining glucose in the supernatants was measured using the Amplex Red Glucose/Glucose Oxidase Assay Kit (A22189, Thermo).

Seahorse assays. Extracellular acidification rates (ECAR) and oxygen consumption rates (OCR) were measured using a Seahorse XFe96 Analyzer (Agilent) in XF media (non-buffered RPMI 1640 containing 2 mM glutamine and 1 mM sodium pyruvate (11360070, Thermo) and 25 mM glucose). Two hundred thousand eGFP⁺ FACS-sorted ITK-SYK-expressing T cells per well were spun onto poly-D-lysine (P6403, Sigma)-coated 96-well plates (101085-004, Agilent) and pre-incubated at 37° C. for 45 min in the absence of CO₂. Five days before the experiment, ITK-SYK^{CD4-creERT2} and ITK-SYK^{CD4-creERT2}; Pdcd1^{-/-} received a single dose of 2 mg tamoxifen and, depending on the experiment, intraperitoneal injections of anti-PD-L1 antibody (200 μg, BE0101, Bioxcell) or isotype control (200 μg, BE0090, Bioxcell) as indicated in the figure legends.

In vivo metabolic imaging using PET/CT and hyperpolarized ¹³C magnetic resonance imaging. C57BL 6, ITK-SYK^{CD4-creERT2}, or ITK-SYK^{CD4-creERT2}; Pdcd1^{-/-} mice received a single dose of 2 mg tamoxifen. For PET/CT imaging, the mice were injected with [¹⁸F]fluorodeoxyglucose ([¹⁸F]FDG, ˜12 MBq), and tracer uptake was measured after 90 min using a preclinical Siemens Inveon PET/CT system. Splenic standardized uptake values were calculated using the Inveon Research Workplace software. Hyperpolarized ¹³C magnetic resonance spectroscopic imaging of the animals was performed using a preclinical 7T MR scanner (Agilent/GE Discovery MR901 magnet and gradient system, Bruker AVANCE III HD electronics). The animals were injected with hyperpolarized [1-¹³C]pyruvate (250 μL, 80 mM, HyperSense DNP, Oxford Instruments), and both [1-¹³C]pyruvate and [1-¹³C]lactate were recorded in vivo using a static 2D FID-CSI (matrix size 18×12, FOV 30 mm×20 mm, slice thickness 3 mm, flip angle 12°, number of points 128, bandwidth 2000 Hz). T2-weighted images (¹H-RARE, matrix size 150×100, FOV 30 mm×20 mm, slice thickness 1 mm) were recorded for co-registration and determination of the spleen size. [1-¹³C]pyruvate vs. [1-¹³C]lactate peak heights were determined and summed over the spleen region of interest using the in-house developed MATLAB code.

Intracellular flow cytometry and Phosflow of murine cells. Ex vivo: ITK-SYK^{CD4-creERT2} or ITK-SYK^{CD4-creERT2}; Pdcd1^{-/-} mice were intraperitoneally injected with a single dose of 2 mg tamoxifen. At the indicated time points, cells were isolated and immediately fixed in 4% PFA (28906, Thermo), permeabilized using methanol, stained for CD4 (100437, BioLegend), p-AKT^{S473} (48-9715-42, Thermo), p-mTOR^{S2448} (2971, CST), Hexokinase 2 (ab209847, Abcam), Glut 1 (PA1-46152, Thermo), p-S6^{S240/244} (5364S, CST), acetyl-Histone H3 Lys27 (13027, CST), HIF1α (36169, CST), Alexa Fluor 647 p-c-Fos^{S63} (8677,CST), PE p-c-Jun^{S73} (8752, CST), and assessed by flow cytometry. Surface and intracellular antibodies were used in dilutions of 1:300 and 1:100, respectively. The following antibodies were used in 1:300 dilutions as secondary antibodies: PE donkey anti-rabbit IgG (406421, Biolegend) or PE goat anti-mouse IgG (405307, Biolegend). The data were acquired using a FACSCanto II or LSRFortessa flow cytometer (BD Biosciences). FlowJo software was used for data analyses (FlowJo LLC). In vitro: eGFP⁺ FACS-sorted T cells from tamoxifen-injected C57BL 6 and ITK-SYK^{CD4-creERT2}; Pdcd1^{-/-} mice were cultured in vitro in glucose-free DMEM (11966025, Thermo) supplemented with 1% dialyzed FCS (A3382001, Thermo). After four hours, the cells were fixed in 4% PFA and processed as described above. An exemplified gating strategy is provided as Supplementary Figure S1.

Metabolomics. ITK-SYK^{CD4-creERT2} and ITK-SYK^{CD4-creERT2}; Pdcd1^{-/-} mice were intraperitoneally injected with a single dose of 2 mg tamoxifen per mouse. Five days later, the spleen and lymph node-derived single-cell suspensions were sorted by FACS for eGFP⁺. Subsequently, the cells were incubated in glucose-free DMEM (11966025, Thermo) supplemented with 10% dialyzed FCS (A3382001, Thermo) and 11 mM uniformly labeled [U¹³C]glucose (CLM-1396, Cambridge Isotope Laboratories). After three hours in vitro culture, the cells were washed with ice-cold 0.9% NaCl and resuspended in 1 mL ice-cold 80% methanol:water solution (v/v, 51140, Thermo and AE71.1, Carl Roth), including internal standards, followed by incubation for eight hours at −80° C. The cell suspension was centrifuged at 13,000×g at 4° C. for 10 min, and then the supernatants were dried in a SpeedVac concentrator (Savant, Thermo). Lyophilized samples were reconstituted in 40 μl of LCMS-grade water, vortexed, and centrifuged again for 10 min at 13,000×g at 4° C. Samples were analyzed using an Agilent 1200 series HPLC system interfaced with an ABSciex 5500 hybrid triple quadrupole/linear ion trap mass spectrometer equipped with an electrospray ionization source operating in the positive or negative mode. The Q1 (precursor ion) and Q3 (fragment ion) transitions, the metabolite identifier, dwell times, and collision energies for both positive and negative ion modes were used according to published methods with additional transitions for Applicants' internal standards^{24,67}. Five microliters of sample was injected onto an XBridge Amide HPLC column (3.5 m; 4.6 mm×100 mm, 186004868, Waters, Milford, MA). The mobile phases were run at 400 μl min⁻¹ and consisted of HPLC buffer A (pH=9.0, 95% (vol/vol) water, 5% (vol/vol) acetonitrile, 20 mM ammonium hydroxide, 20 mM ammonium acetate) and HPLC buffer B (100% acetonitrile). The HPLC settings were as follows: from 0 to 0.1 min, the mobile phase was maintained at 85% buffer B; from 0.1 to 3 min, the percentage of buffer B was decreased from 85% to 30%; from 3 to 12 min, the percentage of buffer B was decreased from 30% to 2% and was maintained at 2% for an additional 3 min. At minute 15, the percentage of buffer B was increased again to 85%, and the column was flushed for an additional 8 min with 85% buffer B. MultiQuant (v2.1.1., ABSciex) software was used for data analysis. Metabolite peaks were normalized by cell number and internal standards prior to statistical analyses.

The retention times for all metabolites were verified using individual purified standards from Sigma: (Glycolysis/Gluconeogenesis Metabolite Library (ML0013-1KT), Pentose Phosphate Metabolite Library (ML0012), TCA Cycle Metabolite Library (ML0010)), L-glutathione reduced (G4251), L-glutathione oxidized (G6654), L-serine (S4500), and α-D-glucose 1-phosphate (G6750) using the same chromatographic method. The metabolites were quantified by integrating the chromatographic peak area of the precursor ion.

For measurements without ¹³C labeling, eGFP⁺ FACS-sorted cells from acutely induced ITK-SYK^{CD4-creERT2}; Pdcd1^{-/-} mice were incubated overnight in vitro in glucose/glutamine-free DMEM (A1443001, Thermo) supplemented with 10% dialyzed FCS (A3382001, Thermo) and 11 mM glucose (G8270, Sigma), 11 mM galactose (G0750, Sigma), or 11 mM galactose combined with 2 mM N-acetylcysteine (A7250, Sigma). The next day, the metabolites were isolated and quantified as described above.

For measurement of acetyl-1,2-[¹³C₂]coenzyme A, cells from the indicated genotypes were prepared as previously described. LC-MS/MS analysis was performed with the following modifications: Ten microliters of sample was injected onto an XSelect HSS T3 XP Column, 100 Å, 2.5 μm, 2.1 mm×100 mm (186006151, Waters). The mobile phases were run at 350 μl min⁻¹ and consisted of HPLC buffer A (20 mM ammonium hydroxide, 20 mM ammonium acetate in water (pH=9)), and HPLC buffer B (20 mM ammonium acetate in methanol). The HPLC settings were as follows: from 0 to 1 min, the mobile phase was maintained at 2% buffer B; from 1 to 6 min, the percentage of buffer B was increased from 2% to 80%, from 6 to 9 min, the percentage of buffer B was increased from 80% to 100%, and was maintained at 100% for an additional 4 min. At minute 13, the percentage of buffer B was decreased again to 2% and the column was flushed for an additional 5 min with 2% buffer B. The used transitions are listed in Table 1.

Transitions used were shown in Table 1:

TABLE 1

| | Q1 | Q3 | DP | EP | CE | CXP |
|---|---|---|---|---|---|---|
| [1,2-¹²C]acetyl-CoA | 403.244 | 78.9 | −40 | −10 | −100 | −11 |
| [1,2-¹²C]acetyl-CoA | 403.244 | 728.1 | −40 | −10 | −16 | −13 |
| [1,2-¹³C]acetyl-CoA | 404.305 | 78.9 | −45 | −10 | −92 | −7 |
| [1,2-¹³C]acetyl-CoA | 404.305 | 134 | −45 | −10 | −30 | −13 |

To establish the method, the following purified standards were purchased: acetyl-1,2-[¹³C₂] coenzyme A lithium salt (658650, Sigma), acetyl coenzyme A sodium salt (A2056, Sigma), and coenzyme A Hydrat (C4282, Sigma).

LC-MS/MS analysis of histone PTMs. ITK-SYK^{CD4-creERT2} and ITK-SYK^{CD4-creERT2}; Pdcd1^{-/-} mice received a single dose of 2 mg tamoxifen. Five days later, cells were isolated from the spleen, and eGFP⁺ cells were sorted by FACS and cultured in glucose-free DMEM (Thermo, 11966025) supplemented with 10% dialyzed FCS (A3382001, Thermo) and 1 mM [U¹³C]glucose (CLM-1396, Cambridge Isotope Laboratories). After four hours in vitro culture, the cells were washed once with PBS and snap-frozen in liquid nitrogen. For histone post-translation modification (PTM) analysis, approximately 1 million cells were extracted with acid. The pelleted cells were resuspended in 100 μL of 0.2 M H₂SO₄ and histones were extracted by rotating overnight at 4° C. Cell debris was removed by centrifugation at 20,817×g for ten minutes at 4° C. Histones were precipitated by adding trichloroacetic acid (85183, Thermo) to a final concentration of 26%. The tubes were mixed and incubated at 4° C. for two hours and spun at 20,817×g for 15 min. Pellets were washed thrice with ice-cold 100% acetone (AA22928-K2, VWR) (5 min rotation at 4° C., 15 min centrifugation at 20,817×g at 4° C. between washes), dried for 15 min at room temperature, resuspended in 20 μl of 1×Laemmli sample buffer per million cells, and boiled at 95° C. for 5 min. Samples were stored at −20° C. until further use. 4%-20% pre-cast polyacrylamide gels (43277.01, Serva) have been used to separate the histones corresponding to 0.5*10⁶ cells. Gels were briefly stained with InstantBlue Coomassie Protein Stain (ab 119211, Abcam). For targeted mass spectrometry analysis, histone bands were excised, washed once with LCMS-grade water (1153331000, Sigma), and de-stained twice (or until transparent) by incubating for 30 min at 37° C. with 200 μl of 50% acetonitrile (ACN) (8825.2, Carl Roth) in 50 mM NH₄HCO₃ (T871.1, Carl Roth). Gel pieces were then washed twice with 200 μl LCMS-grade water and twice with 200 μl of 100% ACN to dehydrate them. Histones were in-gel acylated by adding 20 μl of propionic anhydride (175641, Sigma) and 40 μl of 100 mM NH₄HCO₃. After five minutes, 140 μl of 1 M NH₄HCO₃ was slowly added to the reaction. The pH was confirmed to be approximately 7 for each sample (in cases where the reaction was acidic, a few microliters of 1 M NH₄HCO₃ were added). The samples were incubated at 37° C. for 45 min at 550 rpm. Afterwards, samples were washed five times with 200 μl of 100 mM NH₄HCO₃, four times with 200 μl of MS-grade water, and four times with 200 μl of 100% ACN. They were spun down briefly, and all the remaining ACN was removed. Gel pieces were rehydrated in 50 μl of trypsin solution (25 ng/mL trypsin in 100 mM NH₄HCO₃) (V5111, Promega) with 1 μl spike tides (SPT-ME-TQL, JPT Peptide Technologies) and incubated at 4° C. for 20 min. After the addition of 150 μl of 50 mM NH₄HCO₃, histones were in-gel digested overnight at 37° C. and 550 rpm. Peptides were sequentially extracted by incubating for 10 min at room temperature with 150 μl of 50 mM NH₄HCO₃, twice with 150 μl of 50% ACN (in LCMS-grade water) 0.1% trifluoroacetic acid (TFA) and twice with 100 μl of 100% ACN. During each of the washing steps, the samples were sonicated for 3 min in a water bath followed by a brief spin down. The obtained peptides were dried using a centrifugal evaporator and stored at −20° C. until resuspension in 30 μl of 0.1% TFA. For desalting, peptides were loaded in a C18 Stagetip (prewashed with 20 μl of methanol followed by 20 μl of 80% ACN and 0.1% TFA, and equilibrated with 20 μl of 0.1% TFA), washed twice with 20 μl 0.1% TFA, and eluted three times with 10 μl of 80% ACN and 0.25% TFA. Flow through obtained from loading of peptides in C18 was further desalted with TopTip Carbon (TT1CAR.96, Glygen) by loading the flow through three times (prewashed thrice with 30 μl of 100% ACN followed by equilibration thrice with 30 μl of 0.1% TFA), washed 5 times with 30 μl of 0.1% TFA, and eluted thrice with 15 μl of 70% ACN and 0.1% TFA. Eluted peptides from both desalting steps were combined and evaporated in a centrifugal evaporator, resuspended in 17 μl of 0.1% TFA, and stored at −20° C. The resuspended samples were injected into an Ultimate 3000 RSLCnano system (Thermo) separated in a 25-cm Aurora column (Ionopticks) with a 50-minute gradient from 6 to 43% of 80% acetonitrile in 0.1% formic acid with a 50-minute gradient from 5 to 60% acetonitrile in 0.1% formic acid. The effluent from the HPLC was directly electrosprayed into a Qexactive HF (Thermo) operated in data-dependent mode to automatically switch between full scan MS and MS/MS acquisition. Survey full-scan MS spectra (from m/z 250-1600) were acquired with a resolution of R=60,000 at m/z of 400 (AGC target of 3×10⁶). The 10 most intense peptide ions with charge states between 2 and 5 were sequentially isolated to a target value of 1×10⁵, and fragmented at 27% normalized collision energy. Typical mass spectrometric conditions were as follows: spray voltage, 1.5 kV; no sheath and auxiliary gas flow; heated capillary temperature, 250° C.; ion selection threshold, 33.000 counts. Peak integration was performed using Skyline⁶⁸. Quantified data were further analyzed in R using a formula published previously⁶⁹.

ChIP-seq. ChIP-seq was performed as previously described⁷⁰, but with the inclusion of a spike-in strategy to allow for relative quantification of the detected ChIP signal in control versus experiment⁹¹. Briefly, ITK-SYK^{CD4-creERT2} or ITK-SYK^{CD4-creERT2}; Pdcd1^{-/-} mice were injected with 2 mg tamoxifen. Five days later, 300,000 cells from the spleens were FACS-sorted for eGFP⁺, immediately cross-linked (1% formaldehyde, 10 min room temperature), lysed in 100 μl Buffer-B-0.3 (50 mM Tris-HCl, pH 8.0, 10 mM EDTA, 0.3% SDS, 1×protease inhibitors, Roche), and sonicated in a microtube (C300010011, Diagenode) using a Bioruptor Pico device until most of the DNA fragments were 200-500 base pairs long (settings: temperature 4° C., 20 cycles with 30 seconds On/30 seconds Off). After shearing and centrifugation at 4° C. and 12,000×g for 10 minutes, the supernatant was diluted 1:1 with dilution buffer (1 mM EGTA 300 mM NaCl, 2% Triton x-100, 0.2% sodium deoxycholate, 1×protease inhibitors, Roche). Sonicated chromatin, supplemented with 50 ng of Drosophila spike-in chromatin (53083, Active Motif), was then incubated for 4 h at 4° C. on a rotating wheel with 2 μg of H3K27ac (C15410174, Diagenode) and 1 μg H2Av (61686, Active Motif) antibodies conjugated to 15 μl of Protein-G Dynabeads (10003D, Thermo). Beads were washed five times with Buffer A (10 mM Tris-HCl, pH 7.5, 1 mM EDTA, 0.5 mM EGTA, 1% Triton X-100, 0.1% SDS, 0.1% Na-deoxycholate, 140 mM NaCl, 1×protease inhibitors) and once with Buffer-C(10 mM Tris-HCl, pH 8.0, 10 mM EDTA). Beads were then incubated with 70 μl elution buffer (0.5% SDS, 300 mM NaCl, 5 mM EDTA, 10 mM Tris HCl pH 8.0) containing 2 μl of proteinase K (20 mg/ml) for 1 h at 55° C. and 8 h at 65° C. to reverse formaldehyde crosslinking, and the supernatant was transferred to a new tube. After adding 30 μl elution buffer to the beads again, the eluates were combined and incubated with another 1 μl of proteinase K for one hour at 55° C. Finally, DNA was purified using SPRI AMPure XP beads (A63880, Beckman Coulter) (1:2 sample-to-beads ratio). Purified DNA was used as input for library preparation using the MicroPlex Library Preparation Kit v2 (C05010012, Diagenode) and processed according to the manufacturer's instructions. Libraries were quality controlled by Qubit and Agilent DNA Bioanalyzer. Deep sequencing was performed on HiSeq 1500 systems according to the standard Illumina protocol for 50 bp single-end reads. The reads were aligned to the mouse genome (mm9) and Drosophila genome (dm6) using the Bowtie2 alignment package⁷¹. Aligned reads were sorted and indexed using Samtools (v1.11)⁷². Sambamba (v0.8.0) was used to filter out unmapped, multi-mapped and duplicate reads⁷³. bamCoverage from deepTools (v3.3.2) was used with spike-in (Drosophila) reads as a normalization factor to extract bigwig files for visualization of the data⁷⁴. For differential analysis, the peaks were called using the MACS2 package (v2.2.7.1)⁷⁵. DiffBind (v2.6.6.2) was used for differential analysis using the built-in spike-in (Drosophila) normalization option. GSEA for ChIP-seq peak data was performed using the chip-enrich package (v2.0.1)⁴⁸.

Cell culture. Unless otherwise indicated, murine cells were cultured in DMEM containing 20% FCS. The following compounds were used for the in vitro experiments: glycolysis inhibitor 2-deoxy-D-glucose (D8375, Sigma), mTOR inhibitor Torin-1 (S2827, Selleckchem), HIF1α inhibitor PX-478 2HCl (S7612, Selleckchem), ATP citrate lyase inhibitor BMS-303141 (4609, Tocris), fatty acid synthase inhibitor FT113 (S6666, Selleckchem), and BET-p300/CBP dual inhibitor NE02734 (S9648, Selleckchem). Inhibitors were dissolved in DMSO or water, and the cell culture medium was supplemented with the indicated concentrations of the compound or DMSO/water (as control).

Torin-1 inhibitor treatment in vivo. For in vivo treatment with the mTORC1/2 inhibitor Torin-1, NSG recipient mice received 1×10³ eGFP⁺ FACS-sorted T cells from an ITK-SYK^{CD4-creERT2}; Pdcd1^{-/-} mice, which had been injected with a single dose of 2 mg tamoxifen (T5648, Sigma) five days earlier. Three days after transplantation, the NSG mice received Torin-1 (S2226, Selleckchem) or vehicle (20% NMP+50% PEG400 in ultra-pure water, 494496 and 202398, Sigma) by intraperitoneal injection (10 mg per kg per day), 5 days a week.

CRISPR/Cas9 mediated deletion of Acly. CRISPR-Cas9 ribonucleoproteins (RNPs) were assembled from the crRNA:tracrRNA duplexes and Alt-R Sp Cas9 nuclease (U.S. Pat. Nos. 1,081,059 and 1,072,534, IDT) according to the manufacturer's recommendations. Briefly, equimolar amounts of crRNA and tracrRNA were mixed and resuspended in IDTE buffer at a concentration of 44 μM. This mixture was heated to 95° C. for 5 min and allowed to cool to room temperature before adding 36 μM Cas9 enzyme. A resting phase of 20 minutes at room temperature followed, before the RNPs were used for electroporation. ITK-SYK^{CD4-creERT2}; Pdcd1^{-/-} mice received a single injection of 2 mg tamoxifen. Five days later, eGFP⁺ cells from the spleen were sorted by FACS. 2×10⁶ cells were washed twice with PBS and resuspended in 80 μl P3 solution (V4XP-3032, Lonza), 20 μl RNPs and 1 μl Alt-R Cas9 Electroporation enhancer (1075916, IDT). Cells were electroporated with a 4D-Nucleofector device (AAF-1002B, Lonza) with pulse code EH115. Immediately after electroporation, 1 mL pre-warmed culture media was added to the cuvettes, and cells were kept at 37° C. for ten minutes. Afterwards, cells were washed twice and 1×10⁶ eGFP⁺ cells were intravenously injected into wild-type recipient mice with constitutive Cas9 expression (026179, Jackson Laboratory) to avoid the immunogenicity of Cas9 protein. The following crRNAs were used: GTTCAATGAGAAAGTTCTTG (SEQ ID NO: 1) for Acly (Mm.Cas9.ACLY.1.AJ) and negative control crRNA #1 (1072544, IDT).

Isolation of primary human CTCL cells. This study was approved by the Northwestern University Institutional Review Board. Patients provided informed consent for inclusion in the study. Clinical and demographic information on human subjects are listed in Table 2 (below). Peripheral blood mononuclear cells (PBMCs) were isolated from the blood of patients with leukemic CTCL by Ficoll-Hypaque gradient centrifugation (17144003, Cytiva). Leukemic cells were sorted by FACS (FACSAria 5, BD Biosciences) using cell surface markers that uniquely identified the neoplastic clones. If the antibody to TCRVβ was available, Applicants isolated the CD3⁺ TCRVβ⁻CD8⁻ population. If not, Applicants isolated CD3⁺CD26⁻CD8⁻ cells. Applicants found that the mutational spectra of cells were similar, regardless of the method of isolation. The resulting CTCL cells had a median purity of >90%. The following antibodies were used: Pacific blue CD3 (317313, BioLegend), APC CD3 (317318, BioLegend), PerCPCy5.5 CD8 (45-0088-42, eBioscience), PE CD26 (302705, Biolegend), PE TCR Vβ2 (130-110-095, Miltenyi), FITC TCR V013 (11-5792-41, eBioscience), PE TCR Vβ14 (130-108-804, Miltenyi), and PE TCR Vβ17 (1M2048, Beckman Coulter). For in vitro experiments, CTCL cells and T cells from healthy human donors were cultured in RPMI medium with 10% FCS (Gemini) and 1% penicillin/streptomycin (Gibco). The tumor burden for each patient was determined using well-validated clinical markers, taking whichever value was higher between Sezary cell counts or the CD26 negative cell number by clinical flow cytometry. These were analyzed in conjunction with white blood cell and absolute lymphocyte counts.

TABLE 2
Clinical and demographic information
of human subjects with CTCL.

| | | | Age at | |
|---|---|---|---|---|
| Patient ID | Ethnicity | Sex | diagnosis | Diagnosis |
| NU114 | Caucasian | M | 56 | Leukemic MF |
| NU115 | Caucasian | F | 57 | SS |
| NU134 | Hispanic | F | 46 | SS |
| NU142 | Caucasian | F | 79 | Leukemic MF |
| NU153 | Caucasian | F | 55 | SS |
| NU16 | Caucasian | M | 57 | Leukemic MF |
| NU161 | Caucasian | M | 59 | Leukemic MF |
| NU201 | Hispanic | M | 66 | Leukemic MF |
| NU208 | Caucasian | M | 64 | SS |
| NU215 | Caucasian | F | 79 | SS |
| NU228 | African | F | 65 | SS |
| NU229 | Caucasian | M | 38 | SS |
| NU253 | Caucasian | F | 75 | SS |
| NU296 | Caucasian | F | 55 | SS |
| NU30 | Caucasian | F | 63 | SS |
| NU334 | Caucasian | F | 69 | SS |
| NU51 | Caucasian | M | 72 | SS |
| NU54 | Caucasian | M | 83 | SS |
| NU55 | Caucasian | F | 72 | SS |
| NU56 | Caucasian | F | 67 | SS |
| NU62 | Caucasian | F | 77 | Leukemic MF |
| NU64 | Caucasian | M | 64 | SS |
| NU78 | Caucasian | F | 57 | SS |
| NU8 | Caucasian | M | 71 | SS |
| NU80 | Caucasian | F | 89 | SS |

Ethnicity, sex, age, and diagnosis of human patients studied.
M, male;
F, female;
MF, Mycosis Fungoides;
SS, Sezary Syndrome

DNA sequencing. Genomic DNA was extracted using a QIAamp Micro Kit (56304, Qiagen). DNA libraries were prepared using a KAPA Hyper Prep Kit (KK8504, KAPA Biosystems), and 150 bp paired-end sequencing was performed on an Illumina HiSeq 2000. Sequencing reads were aligned to the human genome (hg19) using the Burrows-Wheeler Aligner⁷⁶.

Somatic copy number calling. To identify somatic copy number aberrations (SNAs) in whole genome sequencing data from primary CTCL cells, Applicants utilized Patchwork (v2.4) with a window size of 10,000 bp. For quality control, Applicants excluded calls with discordant log 2 read ratios and delta B-allele frequency (BAF) or high-frequency-calls in GnomAD, as previously described⁷⁷⁻⁷⁹.

RNA-seq human and TCR a/P sequence analyses. RNA was extracted using the RNeasy Plus Micro Kit (74034, Qiagen). RNA quality was assessed with a Bioanalyzer 2100 (Agilent Technologies) and RNA was quantified using a Qubit RNA HS assay (Q3285, Thermo). cDNA libraries were generated using SMARTseq v4 (634891, Clontech) and Nextera XT (FC-131-1024, Illumina) and sequenced on an Illumina HiSeq with a read length 150 bp paired-end reads. The sequencing reads were aligned using STAR (v2.4.2); gene-specific transcripts were quantified using HT-Seq (v0.6.0); and differentially expressed transcripts were identified using DESeq2 (v1.30.1)⁶². MiXCR software was used (v2.1.10)⁸ to identify T cell receptor sequences from RNA-seq data.

Phosflow analysis of human cells. Malignant CTCL cells were sorted by FACS as described above. CD4⁺ T cells were immunomagnetically isolated from healthy donor-derived PBMCs (11331D, Thermo Scientific). Next, the cells were stimulated with anti-CD3/anti-CD28 beads (1:1 ratio, 11132D, Thermo) for 30 min, washed once with PBS, and subsequently fixed and permeabilized (GAS004, Invitrogen). Phosflow staining was performed with anti-p-S6S^{240/244} (5364S, CST) and secondary AlexaFluor647 anti-rabbit IgG Fab2 (4414S, CST) antibodies in dilutions of 1:100. Cells were analyzed using an LSRII flow cytometer (BD Biosciences).

Experiments with Jurkat T cells. Jurkat cells (clone E6-1; ATCC) and Raji cells (CCL-86, ATCC) were cultured in RPMI with 10% FCS (Gemini) and 1% penicillin/streptomycin (Gibco). For p-c-Jun^{S73} experiments, Jurkat cells transduced with a lentiviral vector encoding PDCD1 or an empty vector control were co-cultured at a 1:1 ratio with PD-L1 overexpressing Raji cells with or without anti-CD3/CD28 beads (11161D, Thermo) for one hour. Fixable live dead staining was used (L34960, Thermo) and the cells were fixed (554722, BD). Cells were then permeabilized with cold methanol, washed, and stained with Alexa Fluor 488 conjugated p-c-Jun^{S73} antibody (12714, CST) in a 1:100 dilution prior to FACS analysis. For AP-1 reporter assays, cells were transduced with a retrovirus encoding an AP-1 fluorescent reporter construct (Addgene #118095) with near-infrared fluorescent protein (iRFP) as the fluorescent reporter. AP-1 reporter Jurkats were then transduced with PDCD1 or an empty vector control and co-cultured at a 1:1 ratio with PD-L1 Raji cells with or without anti-CD3/CD28 beads. After 24 hours, AP-1 reporter activity was determined by FACS.

Glucose uptake assay in human cells Malignant CTCL cells were isolated, and CD4⁺ T cells from healthy donors were isolated as described above. The cells were cultured in vitro with anti-CD3/anti-CD28 beads (1:1 ratio, 11132D, Thermo). The medium was supplemented with the fluorescent glucose analogue 2-NBDG (100 μg/ml, 600470, Cayman). At the indicated time points, the cells were processed according to the manufacturer's protocol (600470, Cayman) and analyzed on an LSRII flow cytometer (BD Biosciences).

Inhibitor treatment of human cells in vitro Malignant CTCL cells and CD4⁺ T cells from healthy donors were isolated as previously described. Next, the cells were stained with Cell Division Tracker Kit (423801, BioLegend) and cultured in vitro with anti-CD3/anti-CD28 beads (1:1 ratio, 11132D, Thermo), 2-DG (S4701, Selleckchem) in the indicated concentrations, everolimus (HY-10218, MedChemExpress), BMS-303141 (SML0784, Sigma), or DMSO vehicle control. After 6 days, the cells were analyzed by flow cytometry, and the division index was determined using the FlowJo proliferation tool (v10.6, FlowJo LLC).

Preparation of mouse and human ATAC-seq libraries. Ex vivo: For mouse ATAC-seq, ITK-SYK^{CD4-creERT2}, or ITK-SYK^{CD4-creERT2}; Pdcd1^{-/-} mice received a single dose of 2 mg tamoxifen. Five days after injection, single-cell suspensions were generated from the spleens and 50,000 eGFP⁺ FACS-sorted cells were used for the transposase reaction. For human ATAC-seq, 50,000 malignant cells from CTCL patients with and without PDCD1 mutation were sorted by FACS as described above. The transposase reaction was performed as previously described⁸¹. In brief, 50,000 cells were pelleted and resuspended in lysis buffer (10 mM Tris HCl pH 7.4, 10 mM NaCl, 3 mM MgCl₂, 0.1% NP-40 [11332473001, Roche], 0.1% Tween-20 [P1379, Sigma], and 0.01% digitonin [G944A, Sigma]). Lysis was performed on ice for 3 min, before washing with 10 mM Tris HCl pH 7.4, 10 mM NaCl, and 3 mM MgCl₂ buffer containing 0.1% Tween-20. The nuclei were pelleted, and transposition (Tagment DNA Enzyme I, 15027865, Illumina) was performed at 37° C. for 30 min with shaking at 1,000 rpm in 10 mM Tris-HCl pH 7.6, 5 mM MgCl₂, 10% dimethyl formamide (D4551, Sigma), PBS, 0.1% Tween-20, 0.01% digitonin. DNA was column-purified (T1030L, NEB), and the transposed DNA was amplified as previously described⁸¹ in 50 μl reaction volumes with a modified version of custom-designed primers⁴⁴. The reaction was monitored after 4 cycles with qPCR using 5 μl PCR reaction mixture and the same primers in 15 μl total volume (KK4617, Roche). Cycles were added as calculated, then the amplified samples were purified, and size selected using SpeedBeads (GE65152105050250, Sigma) in 22% PEG. Libraries were quality-controlled using Qubit and Bioanalyzer (5067-4626, Agilent), and 76 bp paired-end sequencing was performed on an Illumina NextSeq 500. In vitro: For the ATAC-seq experiments involving inhibitor incubation, single-cell suspensions were generated from spleens of acutely induced ITK-SYK^{CD4-creERT2}, or ITK-SYK^{CD4-creERT2}; Pdcd1^{-/-} mice. Next, the cells were incubated in vitro for three hours in presence of 5 μM ACLY inhibitor BMS-303141 or DMSO. Finally, ITK-SYK expressing eGFP⁺ cells were FACS-sorted and immediately processed for ATAC-seq as described above.

ATAC-seq analysis. For mouse ATAC-seq analysis, Cutadapt (v3.1) was used to remove adapter sequences. Next, reads were aligned to the mm9 genome using Bowtie2 (v2.4.0)⁷¹. Afterwards, duplicates were marked with MarkDuplicates (Picard, v2.24.0) and removed with Samtools (v1.11). To filter properly mapped reads, Applicants used Samtools with the following options: exclude multi-mapped reads (MAPQ<30) and reads with flag 1796 or 1804. Next, Samtools with option “-f 2” was used to filter properly paired reads. Adjustment using the Tn5 shift was performed with alignmentSieve (deepTools (v3.3.2)). Next, Applicants filtered for nucleosome-free reads (0-100 bp) as previously described⁴⁴. Visual inspection after filtering was performed using the bamPEFragmentSize function from deepTools (v3.3.2). Next, Applicants used MACS2 (v2.2.7.1) to call ATAC-seq peaks with the following parameters: -nomodel-nolambda-keep-dup auto-call-summits⁷⁵. HINT-ATAC was used to calculate transcription factor profiles based on JASPAR version 2020^{45,46}. chromVAR (v1.14.0) was used to calculate motif scores⁴⁷. Human ATAC-seq analysis was performed similarly with the following modifications: adapter sequences were trimmed using Trimmomatic (v0.36)⁸² and mapping was performed using the hg19 reference genome.

Statistical analysis of biological experiments. All statistical tests were performed using R software (v3.5.0 or higher; R Foundation for Statistical Computing). In each experiment, appropriate statistical tests, including non-parametric tests, were used, as indicated in the figure legends. Multiple comparisons were performed using ANOVA, Tukey's post-hoc test, and corrected p-values. Parametric or non-parametric tests were used based on the data distribution. No specific test for normality or equal variances has been conducted. No statistical methods were used to predetermine sample size estimates. No data were excluded from the analyses, except one animal died before the respective time of analysis. The experiments were not randomized and the Investigators were not blinded to allocation during experiments and outcome assessment. Statistical significance level was set at p<0.05.

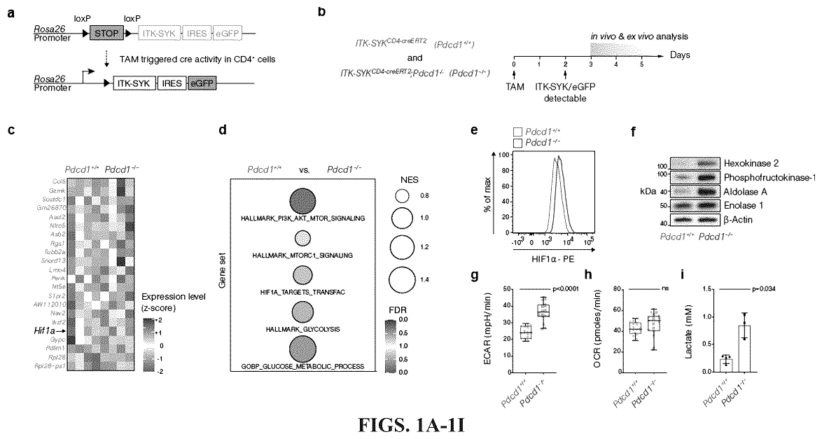

Data availability. All data are available from the corresponding authors upon reasonable request. For mouse data, RNA-sequencing, ATAC-sequencing and ChIP-sequencing data have been deposited in the Gene Expression Omnibus database (GEO) with accessions GSE212832, GSE213180, and GSE1835530. For human data, whole genome sequencing, RNA-sequencing and ATAC-sequencing data for consenting patients is deposited in the database of Genotypes and Phenotypes (dbGaP) under the accession codes phs002456.v1 (for previously published data in Park et al, 2021) and phs003312. Source data for FIGS. 1-11, and 13 have been provided as Source Data files. Raw data from RNAseq and metabolic experiments can be found in the Supplementary Tables 3-14 of Wartewig, T., Daniels, J., Schulz, M. et al. PD-1 instructs a tumor-suppressive metabolic program that restricts glycolysis and restrains AP-1 activity in T cell lymphoma. Nat Cancer 4, 1508-1525 (2023). https://doi.org/10.1038/s43018-023-00635-7, which is hereby incorporated by reference. All other data supporting the findings of this study are available from the corresponding author on reasonable request.

### Example 2: Introduction

T cell non-Hodgkin lymphomas (T-NHLs) represent a heterogeneous group of highly aggressive cancers that typically originate from mature CD4⁺ T cells¹. The therapeutic options for these malignancies are limited which is largely due to their ill-defined molecular pathogenesis¹. However, recent genomic analyses of large T-NHL patient cohorts revealed numerous oncogenic, gain-of-function alterations in the T cell antigen receptor (TCR) signaling pathways. In addition, PDCD1, which encodes for the inhibitory immune receptor PD-1, emerged as a key tumor suppressor in T-NHL². Inactivating mutations in PDCD1 predict an aggressive clinical phenotype, and they portend a poor overall survival in patients². In addition, anti-PD-1 checkpoint inhibitors have been correlated to the emergence of secondary T-NHLs in patients with other primary malignancies^{3,4}. Moreover, clinical trials in T-NHL patients have reported hyperprogression of individual T cell lymphomas—which were apparently still under PD-1 control—after anti-PD-1 treatment (NCT02631746^{5,6}, NCT03075553⁷). While all these clinical observations highlight the critical importance of inhibitory PD-1 signaling in T cell malignancies, the tumor-suppressive mechanisms of PD-1 remain unknown.

In non-transformed T cells, acute PD-1 engagement leads to inhibition of the (PI3K)/AKT axis⁸, and persistent PD-1 signaling triggers T cell exhaustion, a dysfunctional state with specific metabolic and epigenetic characteristics^{9,10}. In addition, RNA-seq analysis from primary human and murine PD-1-deficient T-NHLs² demonstrate that these tumors are characterized by a distinct gene expression that is presumably also shaped by epigenetic mechanisms. Nevertheless, the molecular link between PD-1 signaling, metabolic and epigenetic reprogramming during lymphomagenesis remains unclear.

In non-transformed T cells, acute PD-1 engagement leads to inhibition of the (PI3K)/AKT axis⁸, and persistent PD-1 signaling triggers T cell exhaustion, a dysfunctional state with specific metabolic and epigenetic characteristics^{9,10}. In addition, RNA-seq analysis from primary human and murine PD-1-deficient T-NHLs² demonstrate that these tumors are characterized by a distinct gene expression that is presumably also shaped by epigenetic mechanisms. Nevertheless, the molecular link between PD-1 signaling, metabolic and epigenetic reprogramming during lymphomagenesis remains unclear.

Here Applicants identify PD-1 as a major gatekeeper for the oncogene-triggered metabolic switch to glycolysis. Applicants show that PDCD1 mutations enforce the induction of key metabolic molecules for glucose uptake, metabolization and generation of energy carriers. In addition, PD-1 regulates the ATP citrate lyase (ACLY), which utilizes extramitochondrial citrate to fuel acetyl-CoA pools for histone acetylation and enables aberrant AP-1 activity in the tumor cells. These AP-1 inducing mechanisms are hijacked in aggressive PD-1 deficient lymphomas but not in their PD-1 competent counterparts and link PD-1 signaling to epigenetic reprogramming in T-NHL.

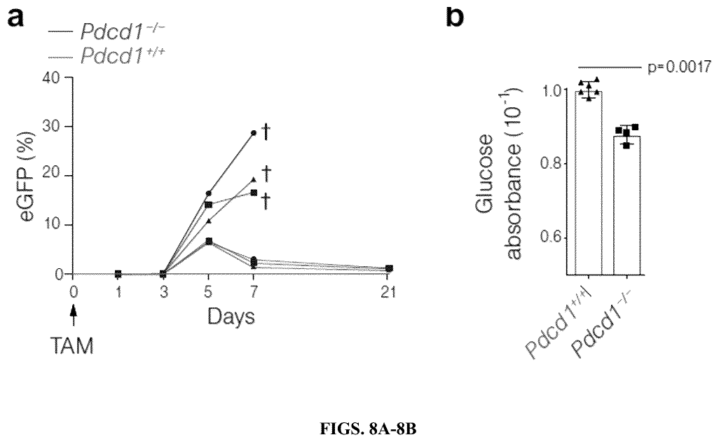

Using tractable mouse models for T-NHL and primary patient samples, Applicants demonstrated that PD-1 signaling suppresses T cell malignancy by restricting glycolytic energy and acetyl-CoA production. In addition, PD-1 inactivation enforced ATP-citrate lyase (ACLY) activity, which generates extramitochondrial acetyl-CoA for histone acetylation to enable hyperactivity of AP-1 transcription factors. Conversely, pharmacological ACLY inhibition impeded aberrant AP-1 signaling in PD-1 deficient T-NHLs and was toxic to these cancers. Applicants' data uncover genotype-specific vulnerabilities in PDCD1 mutated T-NHL and identify PD-1 as regulator of AP-1 activity. To dissect T cell lymphoma pathogenesis, Applicants previously engineered a genetically controllable murine model of human T-NHL based on tamoxifen-inducible irreversible expression of the T-NHL oncogene ITK-SYK, which enforces strong oncogenic TCR signaling¹¹, together with an eGFP reporter in individual mature CD4⁺ T cells in vivo (ITK-SYK^{CD4-creERT2} mice; for experimental system see FIGS. 1A and 1B)^{11,12}. While the acute expression of ITK-SYK (together with eGFP) in otherwise unperturbed primary T cells can trigger lymphocyte proliferation for a few days, it is unable to induce overt malignancy, which requires the acquisition of additional genetic hits (FIG. 8A)^{11,12}. However, the loss of the tumor suppressor gene Pdcd1 is sufficient to enable immediate unrestricted clonal expansion of lymphomatous T cells upon single oncogene expression that is lethal to the host (FIG. 8A)¹². These cells can transmit the malignant disease to secondary recipients¹². Applicants leveraged this genetically tractable mouse model for human T-NHL to investigate the very early events of T cell transformation.

### Example 3: Loss of Pdcd1 Enables Oncogene-Enforced Glycolysis

To identify the potent PD-1-controlled tumor suppressor programs that prevent T cell transformation upon oncogenic T cell signaling, Applicants induced ITK-SYK expression in vivo in primary CD4⁺ T cells with and without Pdcd1 by injecting tamoxifen into ITK-SYK^{CD4-creERT2} and ITK-SYK^{CD4-creERT2}; Pdcd1^{-/-} mice. Three days after ITK-SYK expression, Applicants purified the oncogene-expressing cells by fluorescence-activated cell sorting (FACS) for RNA sequencing. Among the top 20 differentially expressed genes, Hif1α, a master regulator of cellular metabolism, was specifically upregulated in the absence of Pdcd1 (FIG. 1C, p_{adj}<0.0001). In addition, gene set enrichment analysis (GSEA) revealed an enrichment of multiple gene expression signatures that indicate global activation of the phosphoinositide-3 kinase (PI3K)/AKT-mTOR-HIF1α axis¹³ upon PD-1 loss. Furthermore, enrichment of multiple gene expression signatures suggests enhanced glucose metabolism in the Pdcd1-deficient lymphoma cells compared to their pre-malignant ITK-SYK⁺PD-1⁺ counterparts (FIG. 1D). These gene expression data were corroborated by protein expression analysis of HIF1α and several enzymes that mediate glycolysis, including hexokinase 2, phosphofructokinase-1, aldolase A, and enolase 1 in ex vivo isolated, acute oncogene-expressing T cells from tamoxifen-injected ITK-SYK^{CD4-creERT2} and ITK-SYK^{CD4-creERT2}; Pdcd1^{-/-} mice (FIGS. 1E and 1F)^{14,15}.

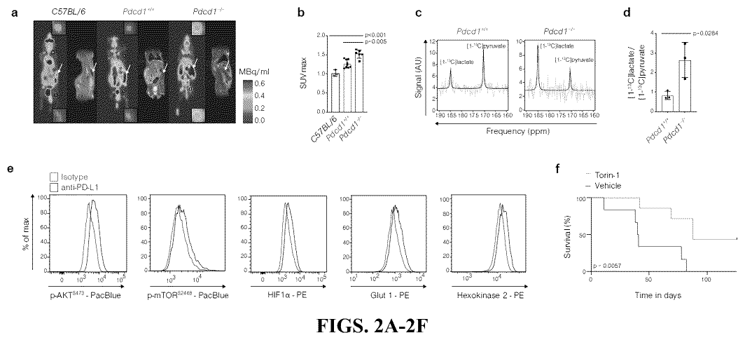

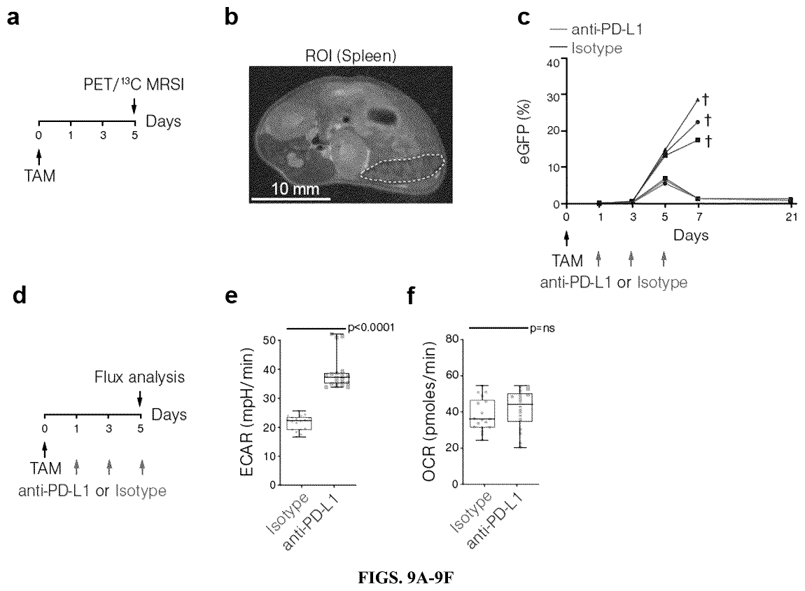

Based on these findings, Applicants next performed direct ex vivo functional analyses of glucose metabolism of acute ITK-SYK-expressing T cells with and without PD-1 function. Upon oncogenic T cell signaling, Applicants detected enhanced glucose uptake in Pdcd1-deficient T cells compared to the wild-type cells (FIG. 8B, p=0.0017). Moreover, extracellular flux analysis revealed an increase in the extracellular acidification rate (ECAR) in the transformed ITK-SYK⁺PD-1− T cells compared to their pre-malignant ITK-SYK⁺PD-1⁻ T cell counterparts, whereas the oxygen consumption rate (OCR) remained unchanged (FIGS. 1G and 1H, p<0.0001 and p=ns). Because the increase in ECAR implies aerobic glycolytic conversion of pyruvate to lactate, Applicants next measured the production of this metabolite. Lactate generation was enhanced in the transformed ITK-SYK⁺PD-1− T cells compared to their pre-malignant counterparts (FIG. 1I, p=0.034). To study glucose metabolism in vivo, Applicants used the glucose analogue tracer ¹⁸F-fluorodeoxyglucose (¹⁸F-FDG) and positron emission tomography (PET). The maximum ¹⁸F-FDG uptake in individual mice was measured in the spleens of both ITK-SYK^{CD4-creERT2} and ITK-SYK^{CD4-creERT2}; Pdcd1^{-/-} mice (FIGS. 2A and 9A). The overall ¹⁸F-FDG uptake and maximum uptake in single voxels was significantly higher in ITK-SYK^{CD4-creERT2}; Pdcd1^{-/-} animals compared to the Pdcd1-proficient ITK-SYK^{CD4-creERT2} mice, demonstrating augmented glucose uptake in vivo (FIG. 2B, p=0.005). To measure glycolytic conversion, Applicants infused hyperpolarized [1-¹³C]pyruvate intravenously into tamoxifen-injected animals and compared the [1-¹³C]lactate/[1-¹³C]pyruvate signal ratios in the spleens using hyperpolarized ¹³C magnetic resonance spectroscopic imaging¹⁶. The [1-¹³C]lactate/[1-¹³C]pyruvate signal ratio was substantially increased in the oncogene-expressing ITK-SYK^{CD4}-^{creERT2}; Pdcd1^{-/-} animals compared to the ITK-SYK^{CD4-creERT2} mice, confirming enhanced lactate production in vivo (FIGS. 2C, 2D, 9A and 8B, p=0.0284).

Altogether, these first sets of genetically controlled in vivo and ex vivo experiments demonstrate that deficiency in Pdcd1 forces T cells with oncogenic signaling to adapt their metabolism to anaerobic glycolysis, characteristic of the Warburg effect that promotes the initiation and progression of most cancer types¹⁷⁻¹⁹.

### Example 4: PD-1 Represses mTOR and HIF1α in Premalignant Cells

To determine how PD-1 inactivation promotes glycolysis, Applicants acutely blocked PD-1 function in ITK-SYK-expressing T cells with anti-PD-L1 checkpoint inhibitors as experimental tools in vivo or ex vivo. This strategy allowed us to control the inactivation of PD-1 signaling in oncogene-expressing T cells in a time-dependent manner. Similar to the genetic deletion of Pdcd1, the injection of anti-PD-L1 into tamoxifen-treated ITK-SYK^{CD4-creRT} mice induced an unrestricted expansion of oncogene-expressing T cells, which rapidly killed the host (FIG. 9C)¹². These acutely PD-1 inhibited cells also switched to glycolysis with an increase in ECAR and no change in their basal OCR (FIGS. 9D-F, p<0.0001 and p=ns). Intracellular flow cytometric analysis directly after acute PD-1 blockade demonstrated that the inactivation of the PD-1 signal resulted in prompt activation of the AKT and mTOR pathways within the oncogene-expressing T cells (FIG. 2E) and in the direct upregulation of HIF1α expression²⁰. Furthermore, Applicants observed an induction of HIF1α transcriptional targets, including the glucose transporter glut 1 and hexokinase 2, which are rate-limiting factors for glucose uptake and glucose utilization, respectively, in normal and malignant lymphocytes (FIG. 2E)²¹. Thus, these key metabolic switches in pre-malignant cells are under the direct negative control of PD-1 tumor suppressor signaling.

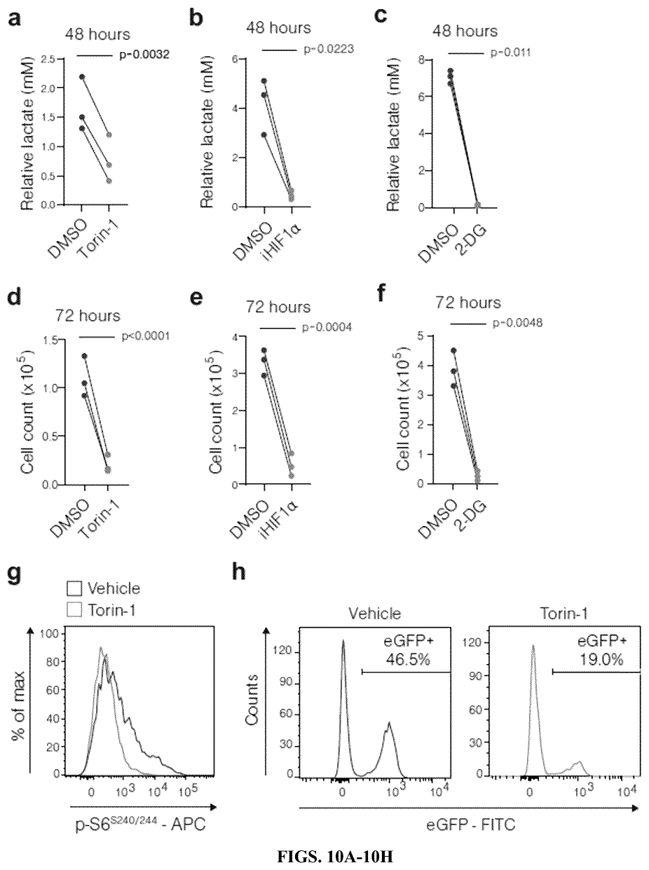

To test the dependency of fully transformed lymphomas with Pdcd1 deletion on the released mTOR-HIF1α-glycolysis cascade, Applicants incubated ITK-SYK⁺PD-1− cells with small molecule inhibitors of mTOR, HIF1α, or glycolysis^{22,23}. The direct inhibition of glycolysis with 2-deoxy-D-glucose (2-DG) reduced the production of lactate, similar results were observed with the inhibition of mTOR or HIF1α (FIGS. 10A-C), and all three treatments were toxic to PD-1 deficient lymphoma cells (FIGS. 10D-F). Moreover, treatment of ITK-SYK⁺PD-1− lymphoma-bearing mice with the mTOR inhibitor Torin-1, which blocks mTOR activity within the tumor cells in vivo (FIG. 10G), significantly prolonged survival (FIGS. 2F and 10H, p=0.0057). Together, these pharmacological studies demonstrate that glycolytic reprogramming is key for the transformation and survival of oncogene-expressing Pdcd1-deficient T cells.

### Example 5: PD-1 Limits Oncogene-Triggered Energy Metabolism

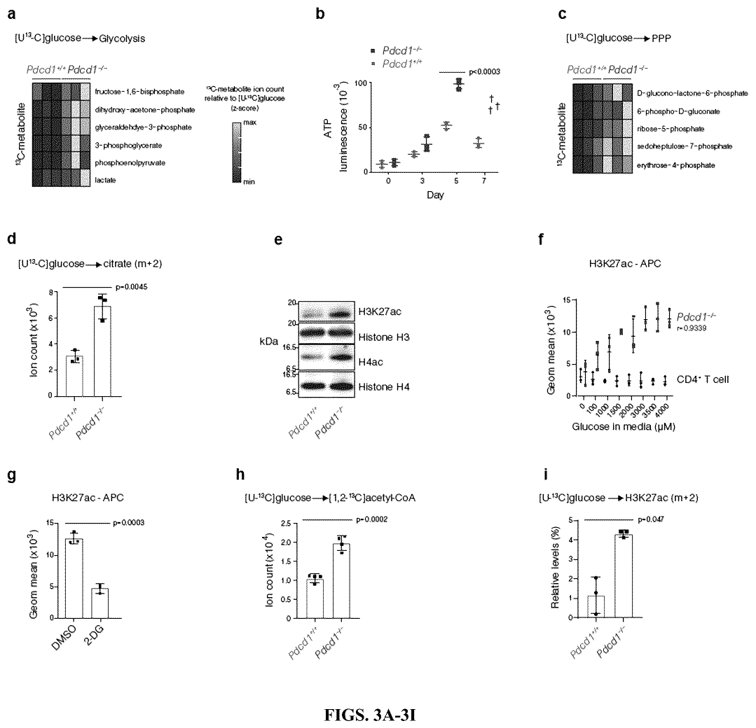

In general, cancer cells utilize the metabolism of glucose to generate ATP for energy supply, rapidly assimilate biomass, and generate signaling molecules that can regulate gene expression¹⁸. To identify the glycolysis-dependent lymphoma-enabling mechanisms in an unbiased manner, Applicants incubated acute oncogene-expressing ITK-SYK⁺PD-1− T cells and their pre-malignant ITK-SYK⁺PD-1⁺ counterparts with uniformly labeled [U-¹³C]glucose ex vivo and performed LC-MS/MS analysis to trace [U-¹³C]glucose-derived metabolites²⁴. The amount of ¹³C labeled glycolysis intermediates, fructose-1,6-bisphosphate, dihydroxy-acetone-phosphate, glyceraldehyde-3-phosphate, 3-phosphoglycerate, phosphoenolpyruvate and lactate, were elevated in Pdcd1-deficient lymphoma cells (FIG. 3A), indicating enhanced glucose usage within the canonical upper glycolytic pathway. This pathway is particularly important for energy production²⁵. Indeed, the inactivation of PD-1 signaling boosts ATP levels (FIG. 3B, p<0.0003), indicating that the loss of PD-1 function can overcome the energy deficit in pre-malignant T cells that is required to fuel overt malignancy.

In addition, Applicants detected increased glucose utilization within the pentose phosphate pathway (PPP) in ITK-SYK⁺PD-1− lymphoma cells, with augmented generation of [U-¹³C]glucose-derived glucono-lactone-6-phosphate, 6-phospho-D-gluconate, ribose-5-phosphate, sedoheptulose-7-phosphate, and erythrose-4-phosphate (FIG. 3C). The overall abundance of the [U-¹³C]glucose-derived tricarboxylic acid cycle (TCA) metabolites only slightly differed between transformed ITK-SYK⁺PD-1− T cells and their pre-malignant ITK-SYK⁺PD-1⁺ counterparts, although the specific levels of citrate (m+2) showed more than 2-fold increase in the absence of Pdcd1 (FIG. 3D, p=0.0045).

### Example 6: PD-1 Facilitates Glycolysis-Dependent Histone-Acetylation

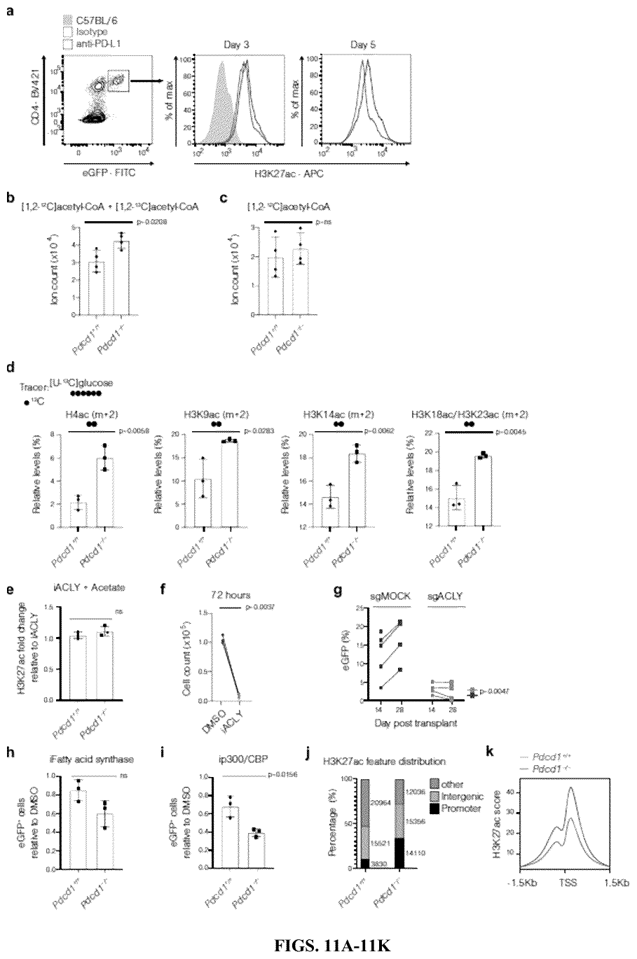

There is increasing evidence that metabolite abundance is also highly relevant for regulation of the tumor epigenome^{26,27}. In particular, histone acetylation is extremely sensitive to the availability of extramitochondrial acetyl-CoA²⁸. In this context, glucose-derived citrate, after export from the mitochondrion, is converted by ATP-citrate lyase (ACLY) to oxalacetate and cytosolic acetyl-CoA, which is subsequently used by acetyl-transferases to mediate the acetylation of target proteins. After entering the nucleus, this pool of acetyl-CoA is critical for histone acetyltransferases (HATs) to mediate histone acetylation for chromatin opening^{29,30} Because these pathways can link glucose metabolism to epigenetic regulation, which is frequently altered in T cell malignancy¹, Applicants next studied the effects of PD-1 inactivation on histone acetylation in ITK-SYK oncogene-expressing primary CD4⁺ T cells. Intriguingly, upon oncogenic T cell signaling, both the genetic deletion of Pdcd1 and acute pharmacological blockade of PD-1 triggered a significant increase in histone H4 and H3 lysine 27 (H3K27) acetylation (FIGS. 3E and 11A). Ex vivo incubation of ITK-SYK⁺PD-1− lymphoma cells in increasing concentrations of glucose demonstrated that the level of H3K27 acetylation depends directly on glucose availability (FIG. 3F, r=0.9339)³¹. Conversely, inhibition of glycolysis with 2-DG resulted in a reduction in H3K27 acetylation in Pdcd1 deficient lymphoma cells (FIG. 3G, p=0.0003)³², demonstrating a link between PD-1 inactivation and glucose-dependent histone acetylation in oncogene-expressing T cells.

To explore whether this link involves glycolysis-dependent de novo generation of acetyl-CoA, Applicants directly incubated ITK-SYK-expressing T cells from tamoxifen-injected ITK-SYK^{CD4-creERT2} and ITK-SYK^{CD4-creERT2}; Pdcd1^{-/-} mice ex vivo with [U-¹³C]glucose and studied acetyl-CoA production by targeted LC-MS/MS analysis. Indeed, in the transformed ITK-SYK⁺PD-1− T cells, there was a significant increase in [1,2-¹³C]-acetyl-CoA and total acetyl-CoA ([1,2-¹²C]-acetyl-CoA+[1,2-¹³C]-acetyl-CoA) generation (FIGS. 3H and 11C, p=0.0002 and p=0.0206), whereas the levels of [1,2-¹²C]-acetyl-CoA did not differ (FIG. 11B, p=ns). Next, Applicants isolated histones from these [U-¹³C]glucose-incubated oncogene-expressing primary T cells for high-resolution LC-MS/MS analysis. Applicants observed increased de novo acetylation of histones H3 and H4 with [U-¹³C]glucose-derived ¹³C-acetyl-marks at H3K27, H3K9, H3K14, and H3K18/K23 in the transformed ITK-SYK⁺PD-1− T cells compared to PD-1 proficient ITK-SYK⁺PD-1⁺ cells (FIGS. 3I and 11D, p=0.047)³³. These data indicate that histone acetylation requires glycolysis-dependent acetyl-CoA synthesis after PD-1 inactivation in T cells with oncogenic T cell signaling.

### Example 7: ACLY is a Critical Effector Molecule Downstream of PD-1

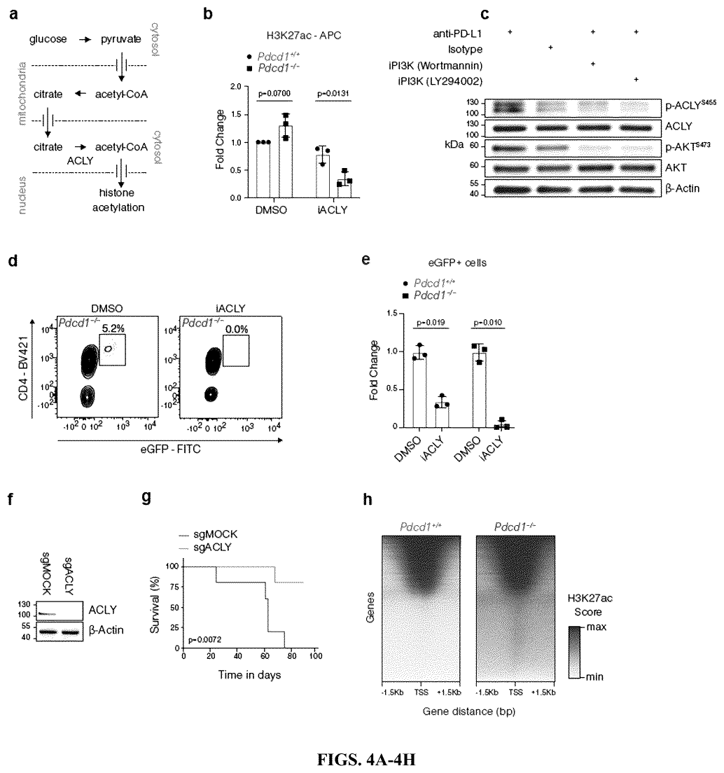

As indicated above, ACLY is the key enzyme that mediates the generation of glucose-derived extramitochondrial acetyl-CoA (FIG. 4A)³⁴. To explore the function of ACLY in PD-1 regulated histone acetylation, Applicants treated tamoxifen-injected ITK-SYK^{CD4-creERT2} mice with anti-PD-L1 or isotype control antibodies and isolated the oncogene-expressing CD4⁺ T-cells. Next, Applicants incubated these cells in vitro with the small molecule ACLY inhibitor BMS-303141 for 3 hours³². This treatment reduced histone acetylation in ITK-SYK⁺ cells in comparison to DMSO vehicle control (FIG. 4B). Notably, the dependency of H3K27ac on ACLY function was significantly higher in ITK-SYK expressing cells with pharmacologically inactivated PD-1 pathway (FIG. 4B, p=0.0131). Consistent with published results, exogenous supplementation of acetate at physiological levels (100 μM) was not able to restore decreased H3K27ac signals in ACLY inhibitor-treated lymphoma cells (FIG. 11E).

The activity of ACLY itself can in principle be regulated by AKT, which can phosphorylate ACLY at serine 455, a key point of control for this enzyme³⁵⁻³⁹. Since PD-1 inactivation results in (PI3K)/AKT signaling in oncogene-expressing T cells (see FIG. 2E), Applicants hypothesized that these events could additionally trigger ACLY activation³⁹. To test this hypothesis, Applicants induced ITK-SYK expression in CD4+ cells by injecting tamoxifen into ITK-SYK^{CD4-creERT2} mice and acutely blocked PD-1 function with anti-PD-L1. Inactivation of PD-1 signaling resulted in the direct activation of ACLY, as measured by phosphospecific p-ACLY^{S455} antibodies (FIG. 4C), and this phosphorylation could be reversed by PI3K inhibition with wortmannin or LY294002 (FIG. 4C)⁴⁰.

To determine whether ACLY activity is required for PD-1-deficient lymphoma cell survival, Applicants next incubated transformed ITK-SYK⁺PD-1− T cells from tamoxifen-injected ITK-SYK^{CD4-creERT2}; Pdcd1^{-/-} mice with an ACLY inhibitor. This treatment killed the ITK-SYK⁺PD-1⁻ lymphoma cells (FIG. 11F, p=0.0037). Importantly, co-cultures with splenocytes demonstrated that ITK-SYK⁺PD-1− T cells were highly sensitive to ACLY inhibition whereas ITK-SYK⁺PD-1⁺ T cells and normal T cells were less affected (FIGS. 4E and F, p=0.019 and p=0.010).

To genetically define whether ACLY is necessary for PD-1 deficient lymphoma growth in vivo, Applicants disrupted Acly in transformed ITK-SYK⁺PD-1⁻ T cells using CRISPR/Cas9-mediated gene editing and transplanted these cells into syngeneic wild-type hosts (FIG. 4F). While the control-edited ITK-SYK⁺PD-1⁻ cells proliferated rapidly in vivo, as measured by eGFP monitoring, and killed all recipient animals as expected in less than 80 days, the ACLY-deficient ITK-SYK⁺PD-1⁻ T cells were unable to expand (FIGS. 11G and 11I, p=0.0072). The acetyl-CoA pool can be utilized for both histone acetylation and other acetyl-CoA dependent pathways such as fatty acid synthesis. To distinguish the importance of these pathways, Applicants utilized pharmacological inhibitors. Applicants' experiments demonstrated that ITK-SYK⁺PD-1⁻ cells were more sensitive to direct inhibition of histone acetylation with inhibitors of the histone acetyltransferase p300/CBP in comparison to their PD-1 competent counterparts. In contrast, inhibition of the fatty acid synthase did not reveal a significant difference (FIGS. 11H and 11I, p=0.0156).

Together, these functional and genetic data demonstrate that loss of the PD-1 tumor suppressor function results in enhanced ACLY activity via enforced (PI3K)/AKT signaling, and that this mechanism is critical for both glucose-dependent histone acetylation and the malignant expansion of PD-1 deficient lymphomatous T cells.

### Example 8: PD-1 Controls Epigenetic Reprogramming and AP-1 Activity

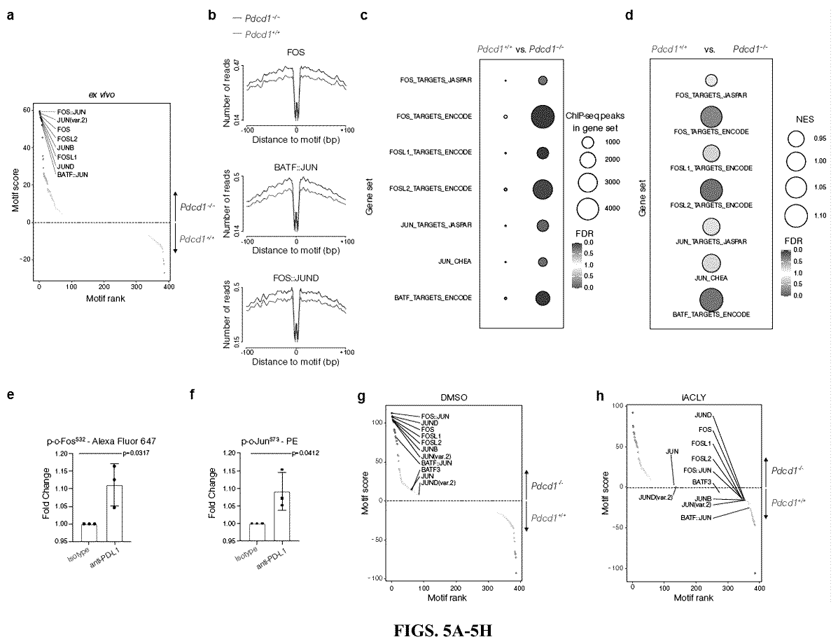

After establishing enhanced histone acetylation as a consequence of Pdcd1 inactivation in oncogene-expressing T cells, Applicants next tested the epigenetic effects of this mechanism on genome regulation using genome-wide assays. Because H3K27 showed the highest dependency on glucose-dependent de novo acetylation, Applicants immunoprecipitated H3K27-acetylated histones from ITK-SYK-expressing T cells with or without PD-1 activity and performed high-throughput chromatin immunoprecipitation sequencing (ChIP-seq⁴¹). Overall, the deletion of Pdcd1 increased H3K27 acetylation in oncogene-expressing T cells, particularly within promoter regions and around gene transcription start sites (TSS), indicating direct effects on transcriptional regulation (FIGS. 4H and 11J and 11K)^{42,43}. To assess how these events would affect chromatin accessibility for transcription factor occupancy, Applicants conducted a genome-wide assay for transposase-accessible chromatin using sequencing (ATAC-seq⁴⁴) and performed transcription factor footprint analysis⁴⁵⁻⁴⁷. Intriguingly, in the PD-1 deficient ITK-SYK-expressing T cells, Applicants observed increased activity scores for the AP-1 family transcription factors c-FOS, FOSL1, FOSL2, c-JUN, JUNB, and BATF (FIGS. 5A and 5B). Next, Applicants tested whether the histones at the AP-1 family target gene sequences are hyper-acetylated in PD-1 deficient ITK-SYK-expressing T cells by performing GSEA using the H3K27ac ChIP-seq dataset (see FIG. 4H; ChIP-Enrich analysis⁴⁸) and the transcription factor target gene sets from the ENCODE, JASPAR, and ChEA databases^{46,49-51}. Applicants detected a significant enrichment of c-FOS, FOSL1, FOSL2, c-JUN, and BATF target genes in H3K27ac ChIP-seq peaks upregulated in the ITK-SYK⁺PD-1⁻ lymphoma cells compared to their pre-malignant ITK-SYK⁺PD-1⁺ counterparts (FIG. 5C). Furthermore, the global expression of AP-1 target mRNAs also increased with a positive enrichment of the c-FOS, FOSL1, FOSL2, c-JUN, and BATF gene sets in transformed ITK-SYK⁺PD-1− cells (FIG. 5D).

To investigate the underlying mechanism for AP-1 hyperactivation, Applicants focused on the top-ranking motif in the PD-1 deficient lymphoma cells, which was the c-FOS:c-JUN heterodimer (see FIG. 5A). Applicants performed additional ex vivo experiments to determine the PD-1 dependent phosphorylation status of c-FOS at serine 32 and of c-JUN at serine 73, respectively. These post-translational modifications are known to increase protein stability, nuclear localization and transcriptional activity^{52,53}. To capture the immediate changes upon PD-1 pathway inactivation, Applicants utilized pharmacological blockage of PD-1 as introduced above (see FIG. 2E). Applicants' Phosflow data revealed a significant increase in p-c-FOS^{S32} and p-c-JUN^{S73} signals in ITK-SYK expressing cells isolated from animals, which had been treated with anti-PD-L1 checkpoint inhibition, suggesting that PD-1 indeed controls the transduction of oncogenic signals to AP-1 family members (FIGS. 5E and 5F, p=0.0317 and p=0.0412).

Applicants asked whether the AP-1 hyperactivity in ITK-SYK⁺PD-1⁻ lymphoma cells is induced by ACLY activity and subsequent inflated acetyl-CoA pools. To address this question, Applicants again isolated ITK-SYK⁺PD-1⁺ and ITK-SYK⁺PD-1⁻ cells from tamoxifen-treated ITK-SYK^{CD4-creERT2} and ITK-SYK^{CD4-creERT2}; Pdcd1^{-/-} animals. This time, Applicants incubated the cells in vitro in presence of the ACLY inhibitor BMS-303141 or DMSO for 3 hours. Next, Applicants FACS-sorted the eGFP⁺ cells and performed ATAC-seq and transcription factor footprint analysis. In agreement with the ex vivo results (see FIG. 5A), Applicants detected AP-1 family members as top motifs enriched in PD-1 deficient cells (FIG. 5G). Strikingly, upon pharmacological inhibition of ACLY, the AP-1 footprints were de-enriched in the Pdcd1 deleted cells in comparison to their counterparts with intact PD-1 pathway (FIG. 511).

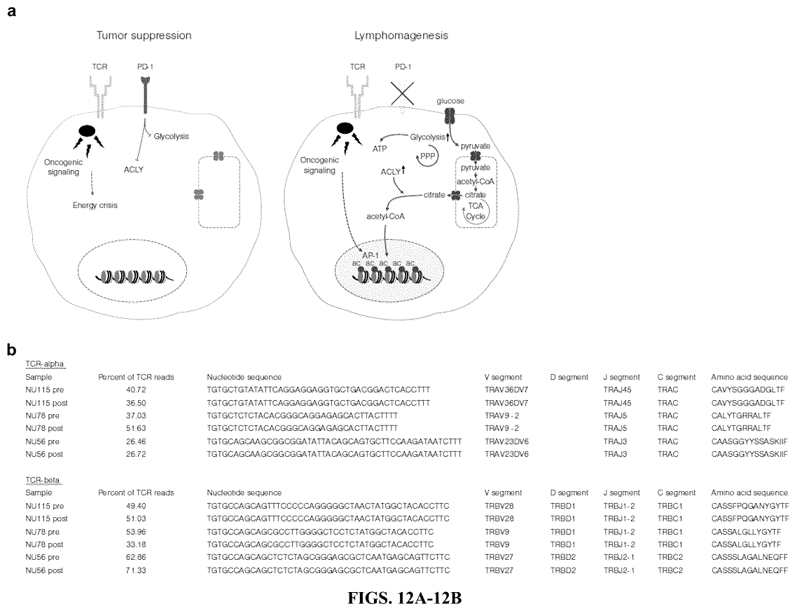

Altogether, these results from Applicants' genetically defined mouse models demonstrate that the loss of PD-1 signaling unleashes ACLY activity and thereby triggers glucose-dependent production of extramitochondrial acetyl-CoA to mediate the de novo acetylation of histones to open chromatin and enable the enhanced activity of oncogenic AP-1 family transcription factors. Importantly, AP-1 hyperactivation in PD-1 deleted lymphoma cells was critically dependent on ACLY activity. Thus, Applicants' results indicate a link between glucose-derived acetyl-CoA availability and selective opening of compact chromatin at AP-1 binding sites, which is mediated by the PD-1 pathway (for a schematic model, see FIG. 12A).

### Example 9: Glycolysis and AP-1 Activation in Human PDCD1 Mutated T-NHL

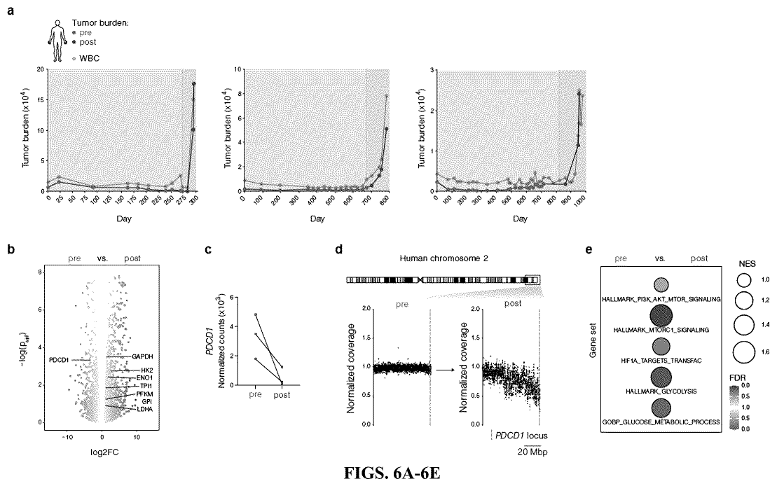

To elucidate whether these PD-1 dependent metabolic and epigenetic changes are also present in human T cell lymphoma, Applicants analyzed leukemic stage primary patient samples from cohorts of clinically annotated T-NHLs of cutaneous origin by isolating malignant lymphoma cells by FACS. Applicants first examined a cohort of patients who experienced rapid disease progression, where the blood tumor burden increased by >400% in less than a one-month period, which Applicants termed “hyperprogression” (FIG. 6A). TCR clonotyping confirmed that post-hyperprogression samples originated from identical T cell clones as the pre-hyperprogression samples (FIG. 12B). Intriguingly, in all three cases, hyperprogressive disease was associated with a profound downregulation of PDCD1 transcripts (FIGS. 6B and 6C, p=4.6E-04, log₂(fold change)=−3.3). Notably, one of these cancers acquired a genetic deletion of PDCD1 upon disease progression (FIG. 6D), whereas the others lost PDCD1 expression by still uncharacterized mechanisms. These pre- and post-hyperprogression lymphomas were used for comparative RNA sequencing and GSEA analysis. Consistent with Applicants' mouse model, Applicants detected a significant enrichment of all signatures that represent enhanced activity of the (PI3K)/AKT-mTOR pathway, enforced HIF1α activity, and increased glucose metabolism in the post-hyperprogression samples with impaired PDCD1 expression compared to pre-hyperprogression samples (FIG. 6E).

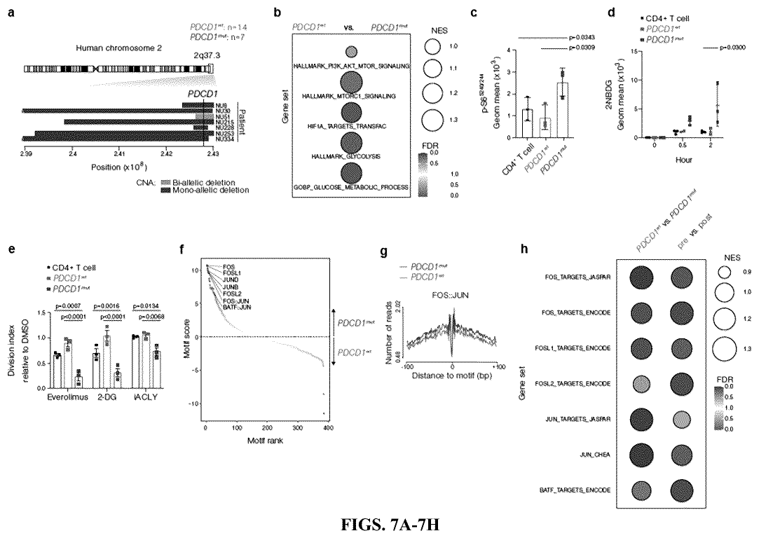

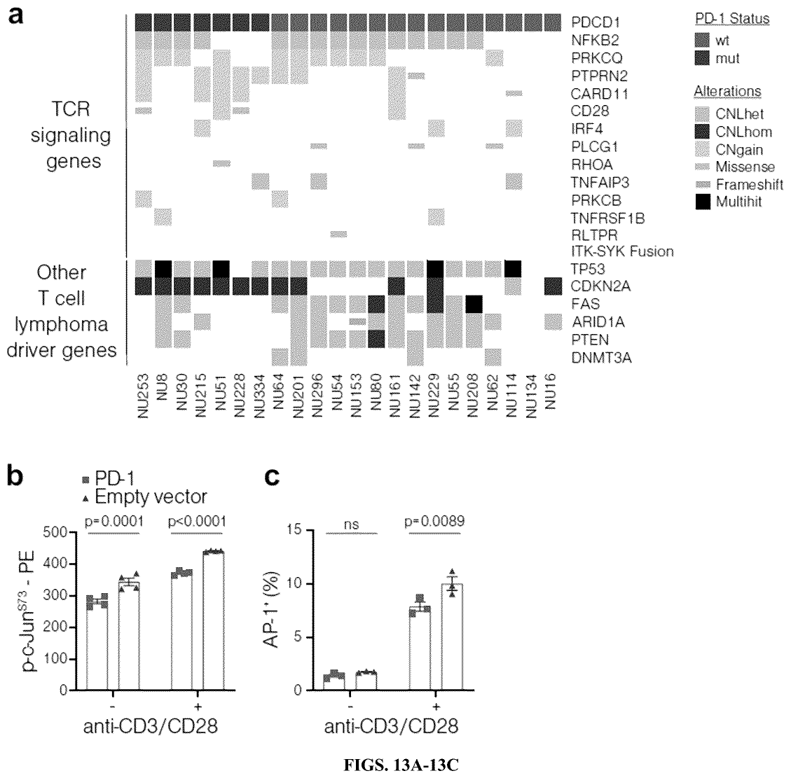

In addition, Applicants studied a second cohort of patients with cutaneous T cell lymphoma (CTCL) that were either PDCD1-wild-type or PDCD1-mutant at initial diagnosis. In line with previously reported frequencies^{2,12}, Applicants detected PDCD1 deletions in seven out of the 21 specimens (FIG. 7A). RNA sequencing and GSEA revealed a significant enrichment of the key gene sets for (PI3K)/AKT-mTOR signaling and HIF1α activation (FIG. 7B) in the PDCD1-mutant cases. Again, Applicants also observed enhanced activity of the glycolysis gene sets in the PDCD1-mutant lymphomas compared to PDCD1 wild-type lymphomas (FIG. 7B). None of the analyzed primary patient samples harbored the rare ITK-SYK fusion oncogene that Applicants used in the mouse to model oncogenic TCR signaling. Instead, the PDCD1-mutant human tumors harbored diverse and numerous oncogenic TCR signaling mutations including in CD28, PLCG1, and RHOA, suggesting the transcriptional effects are not tied to the identity of the oncogene (FIG. 13A). Interestingly, multiple PDCD1-mutant lymphomas carried mutations in another tumor suppressor, CDKN2A (FIG. 13A).

Next, Applicants utilized viable primary patient samples from these two cohorts to functionally assess Applicants' findings on the (PI3K)/AKT-mTOR axis, which is under PD-1 control in normal and malignant T cells, and Applicants' discovery of ACLY as a PD-1 target molecule in T-NHL. First, Applicants probed the impact of PDCD1 deletions on (PI3K)/AKT-mTOR signaling and glucose uptake based on six individual patients, three with mutant PDCD1 and three with wildtype PDCD1. In line with Applicants' findings in the murine model, the PDCD1-mutant lymphoma cells exhibited a significant increase in S6 ribosomal protein phosphorylation, demonstrating enhanced mTOR activity compared to the PDCD1-wild-type cells (FIG. 7C, p=0.0309)⁵⁴. Moreover, uptake of the fluorescent glucose analogue 2-NBDG was also significantly increased in PDCD1-mutant lymphoma cells (FIG. 7D, p=0.03)⁵⁵. To perturb (PI3K)/AKT-mTOR signaling, glycolysis, or ACLY activity in viable PDCD1-wild-type and PDCD1-mutant lymphoma cells, Applicants incubated the six primary patient samples with respective small molecule inhibitors of these pathways. Treatment with the mTORC1 inhibitor everolimus, which is currently in clinical trials for T-NHL therapy (NCT00918333 and NCT01075321), reduced the viability and division of the PDCD1-mutant cancer cells (FIG. 7E). Similarly, inhibition of glycolysis with 2-DG or ACLY blockage with BMS-303141 also significantly impaired cell proliferation in PD-1 deficient lymphomas (FIG. 7E). Importantly, stage-matched PD-1 wild-type tumors were resistant to everolimus, 2-DG and inhibition of ACLY. These data suggest that PD-1 loss confers therapeutic vulnerability to these inhibitors (FIG. 7E).

Finally, Applicants assessed chromatin accessibility in primary cells from three PDCD1-wild-type and three PDCD1-mutant CTCLs by ATAC-seq and performed transcription factor footprint analysis (FIGS. 7F and 7g). Similar to the murine model, the top transcription factor motifs most enriched in the PDCD1-mutant compared to the wild-type were AP-1 family members, including c-FOS, FOSL1, FOSL2, c-JUN, JUNB, and BATF, which Applicants corroborated by functional in vitro assays (FIGS. 7F, 7G, 13B and 13C). Furthermore, at the global transcript level, both PDCD1-mutant and post-hyperprogression T-NHL samples showed a significant enrichment of c-FOS, FOSL1, FOSL2, c-JUN, and BATF target genes (FIG. 7H).

### Example 10: Discussion

Here Applicants identified the key components of the PD-1 tumor suppressor program in T cell lymphoma. Utilizing a combination of genetic mouse models and primary human T-NHL samples, Applicants' results highlight the role of PD-1 as a critical gatekeeper molecule curtailing the glycolytic switch during the neoplastic transformation of a T cell. Applicants provide evidence that metabolic reprogramming via the (PI3K)/AKT-mTOR-HIF1α axis leads to the induction of rate-limiting factors for glucose uptake and metabolization to enforce tumor cell energy production. This tumor suppressive activity of PD-1 specifically suppresses glycolytic reprogramming but not oxidative phosphorylation.

Interestingly, Applicants' here presented data and previous work² indicates that the PD-1 mutation status could serve as a potential biomarker for genotype-specific vulnerabilities in T cell lymphomas. For example, mTOR is currently evaluated as a target in T-NHL with heterogeneous clinical results⁵⁶. Applicants provide evidence that molecular stratification based on the genetic PDCD1 status could help to identify patients who will likely respond to mTOR pathway inhibition. Moreover, Applicants' data suggest enhanced ACLY activity as a selective vulnerability in PDCD1-mutant T-NHLs. Furthermore, Applicants describe a previously unknown link between PD-1 signaling and metabolite-controlled epigenetic reprogramming. Upon PD-1 loss, increased pools of glycolysis-derived citrate permit enhanced acetyl-CoA production. This in turn enforces opening of compact chromatin at AP-1 binding sites, and enhanced transcription of AP-1 target genes. Conversely, pharmacological inhibition of ACLY curtails AP-1 hyperactivation and is toxic to PDCD1-mutant lymphomas. Notably, antigenic stimulation of non-transformed T cells also induces AP-1 activity and enforces AP-1-directed chromatin remodelling⁵⁷. However, in contrast to oncogenic AP-1 activation in T-NHL, this process strictly depends on the external induction of co-stimulatory pathways such as CD28⁵⁷ through still ill-defined mechanisms.

While Applicants' results identify PD-1 as a metabolic gatekeeper for T-NHL energy metabolism and induction of the epigenetic ACLY—AP-1 axis, several questions remain open. On the one hand, it is unclear whether unleashed ACLY activity in PDCD1-mutant lymphomas regulates acetyl-CoA-dependent metabolic pathways apart from histone acetylation, like de novo generation of lipids. On the other hand, it is undefined how PD-1 signaling regulates individual AP-1 family members. While Applicants' data show enhanced phosphorylation of key serine residues within the transactivation domains of c-JUN and c-FOS in the absence of PD-1 signaling, the precise mechanisms that underly these effects remain to be uncovered. In this context, it will also be critical to elucidate how acetyl-CoA abundance can selectively enhance recruitment of AP-1 factors to AP-1:DNA binding sites and whether related pathways are also active in non-transformed T cells and during T cell exhaustion, which is frequently associated with chronic PD-1 signaling and dysregulated AP-1 activity⁵⁸.

Taken together Applicants' findings establish PD-1 as a central regulator for tumor cell energy metabolism and AP-1 activity in T-NHL. Applicants demonstrate that PD-1 restricts glycolysis, glucose-derived acetyl-CoA production and chromatin remodeling. PD-1 deficient T-NHLs acquire epigenetic changes that increase access for AP-1 factors in otherwise compact DNA. This mechanism is highly conserved between human and murine lymphomas. Applicants' results link PD-1 mutations with an AP-1 hyperactivation which has been demonstrated to be a hallmark of aggressive, drug-resistant T-NHLs⁵⁹. Moreover, Applicants' data highlights the (PI3K)/AKT-mTOR-HIF1α/ACLY-axis as a genotype-specific therapeutic vulnerability in aggressive T cell lymphomas with defective PD-1 function, which should be tested in future clinical interventions.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc.

As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Fiore, D., et al. Peripheral T cell lymphomas: from the bench to the clinic. Nat Rev Cancer 20, 323-342 (2020).
2. Park, J., et al. Integrated Genomic Analyses of Cutaneous T Cell Lymphomas Reveal the Molecular Bases for Disease Heterogeneity. Blood (2021).
3. Anand, K., et al. T-cell lymphoma secondary to checkpoint inhibitor therapy. J Immunother Cancer 8(2020).
4. Marks, J. A., Parker, D. C., Garrot, L. C. & Lechowicz, M. J. Nivolumab-associated cutaneous T-cell lymphoma. JAAD Case Rep 9, 39-41 (2021).
5. Rauch, D. A., et al. Rapid progression of adult T-cell leukemia/lymphoma as tumor-infiltrating Tregs after PD-1 blockade. Blood 134, 1406-1414 (2019).
6. Ratner, L., Waldmann, T. A., Janakiram, M. & Brammer, J. E. Rapid Progression of Adult T-Cell Leukemia-Lymphoma after PD-1 Inhibitor Therapy. N Engl J Med 378, 1947-1948 (2018).
7. Bennani, N. N., et al. A Phase II Study of Nivolumab in Patients with Relapsed or Refractory Peripheral T-Cell Lymphoma. Blood 134, 467-467 (2019).
8. Patsoukis, N., Li, L., Sari, D., Petkova, V. & Boussiotis, V. A. PD-1 increases PTEN phosphatase activity while decreasing PTEN protein stability by inhibiting casein kinase 2. Mol Cell Biol 33, 3091-3098 (2013).
9. Sen, D. R., et al. The epigenetic landscape of T cell exhaustion. Science 354, 1165-1169 (2016).
10. Franco, F., Jaccard, A., Romero, P., Yu, Y. R. & Ho, P. C. Metabolic and epigenetic regulation of T-cell exhaustion. Nat Metab 2, 1001-1012 (2020).
11. Pechloff, K., et al. The fusion kinase ITK-SYK mimics a T cell receptor signal and drives oncogenesis in conditional mouse models of peripheral T cell lymphoma. J Exp Med 207, 1031-1044 (2010).
12. Wartewig, T., et al. PD-1 is a haploinsufficient suppressor of T cell lymphomagenesis. Nature 552, 121-125 (2017).
13. Chi, H. Regulation and function of mTOR signalling in T cell fate decisions. Nat Rev Immunol 12, 325-338 (2012).
14. Luo, F., et al. Hypoxia-inducible transcription factor-1alpha promotes hypoxia-induced A549 apoptosis via a mechanism that involves the glycolysis pathway. BMC Cancer 6, 26 (2006).
15. Schodel, J., et al. High-resolution genome-wide mapping of HIF-binding sites by ChIP-seq. Blood 117, e207-217 (2011).
16. Topping, G. J., et al. Acquisition strategies for spatially resolved magnetic resonance detection of hyperpolarized nuclei. MAGMA 33, 221-256 (2020).
17. Vander Heiden, M. G., Cantley, L. C. & Thompson, C. B. Understanding the Warburg effect: the metabolic requirements of cell proliferation. Science 324, 1029-1033 (2009).
18. Liberti, M. V. & Locasale, J. W. The Warburg Effect: How Does it Benefit Cancer Cells?Trends Biochem Sci 41, 211-218 (2016).
19. Warburg, O. [symbol] ber den Stoffwechsel der Carcinomzelle. Die Naturwissenschaften 12, 1131-1137 (1924).
20. Dodd, K. M., Yang, J., Shen, M. H., Sampson, J. R. & Tee, A. R. mTORC1 drives HIF-1alpha and VEGF-A signalling via multiple mechanisms involving 4E-BP1, S6K1 and STAT3. Oncogene 34, 2239-2250 (2015).
21. Muschen, M. Metabolic gatekeepers to safeguard against autoimmunity and oncogenic B cell transformation. Nat Rev Immunol 19, 337-348 (2019).
22. Liu, Q., et al. Development of ATP-competitive mTOR inhibitors. Methods Mol Biol 821, 447-460 (2012).
23. Welsh, S., Williams, R., Kirkpatrick, L., Paine-Murrieta, G. & Powis, G. Antitumor activity and pharmacodynamic properties of PX-478, an inhibitor of hypoxia-inducible factor-1alpha. Mol Cancer Ther 3, 233-244 (2004).
24. Yuan, M., et al. Ex vivo and in vivo stable isotope labelling of central carbon metabolism and related pathways with analysis by LC-MS/MS. Nat Protoc 14, 313-330 (2019).
25. DeBerardinis, R. J. & Chandel, N. S. We need to talk about the Warburg effect. Nat Metab 2, 127-129 (2020).
26. Kinnaird, A., Zhao, S., Wellen, K. E. & Michelakis, E. D. Metabolic control of epigenetics in cancer. Nat Rev Cancer 16, 694-707 (2016).
27. Peng, M., et al. Aerobic glycolysis promotes T helper 1 cell differentiation through an epigenetic mechanism. Science 354, 481-484 (2016).
28. Sivanand, S., Viney, I. & Wellen, K. E. Spatiotemporal Control of Acetyl-CoA Metabolism in Chromatin Regulation. Trends Biochem Sci 43, 61-74 (2018).
29. Wellen, K. E., et al. ATP-citrate lyase links cellular metabolism to histone acetylation. Science 324, 1076-1080 (2009).
30. Campbell, S. L. & Wellen, K. E. Metabolic Signaling to the Nucleus in Cancer. Mol Cell 71, 398-408 (2018).
31. Cluntun, A. A., et al. The rate of glycolysis quantitatively mediates specific histone acetylation sites. Cancer Metab 3, 10 (2015).
32. Li, J. J., et al. 2-hydroxy-N-arylbenzenesulfonamides as ATP-citrate lyase inhibitors. Bioorg Med Chem Lett 17, 3208-3211 (2007).
33. Volker-Albert, M. C., Schmidt, A., Forne, I. & Imhof, A. Analysis of Histone Modifications by Mass Spectrometry. Curr Protoc Protein Sci 92, e54 (2018).
34. Icard, P., et al. ATP citrate lyase: A central metabolic enzyme in cancer. Cancer Lett 471, 125-134 (2020).
35. Lee, J. V., et al. Akt-dependent metabolic reprogramming regulates tumor cell histone acetylation. Cell Metab 20, 306-319 (2014).
36. Martinez Calejman, C., et al. mTORC2-AKT signaling to ATP-citrate lyase drives brown adipogenesis and de novo lipogenesis. Nat Commun 11, 575 (2020).
37. Covarrubias, A. J., et al. Akt-mTORC1 signaling regulates Acly to integrate metabolic input to control of macrophage activation. Elife 5(2016).
38. Migita, T., et al. ATP citrate lyase: activation and therapeutic implications in non-small cell lung cancer. Cancer Res 68, 8547-8554 (2008).
39. Potapova, I. A., El-Maghrabi, M. R., Doronin, S. V. & Benjamin, W. B. Phosphorylation of recombinant human ATP:citrate lyase by cAMP-dependent protein kinase abolishes homotropic allosteric regulation of the enzyme by citrate and increases the enzyme activity. Allosteric activation of ATP:citrate lyase by phosphorylated sugars. Biochemistry 39, 1169-1179 (2000).
40. Vlahos, C. J., Matter, W. F., Hui, K. Y. & Brown, R. F. A specific inhibitor of phosphatidylinositol 3-kinase, 2-(4-morpholinyl)-8-phenyl-4H-1-benzopyran-4-one (LY294002). J Biol Chem 269, 5241-5248 (1994).
41. Mardis, E. R. ChIP-seq: welcome to the new frontier. Nat Methods 4, 613-614 (2007).
42. Mizzen, C. A. & Allis, C. D. Linking histone acetylation to transcriptional regulation. Cell Mol Life Sci 54, 6-20 (1998).
43. Kurdistani, S. K., Tavazoie, S. & Grunstein, M. Mapping global histone acetylation patterns to gene expression. Cell 117, 721-733 (2004).
44. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218 (2013).
45. Li, Z., et al. Identification of transcription factor binding sites using ATAC-seq. Genome Biol 20, 45 (2019).
46. Fornes, O., et al. JASPAR 2020: update of the open-access database of transcription factor binding profiles. Nucleic Acids Res 48, D87-D92 (2020).
47. Schep, A. N., Wu, B., Buenrostro, J. D. & Greenleaf, W. J. chromVAR: inferring transcription-factor-associated accessibility from single-cell epigenomic data. Nat Methods 14, 975-978 (2017).
48. Welch, R. P., et al. ChIP-Enrich: gene set enrichment testing for ChIP-seq data. Nucleic Acids Res 42, e105 (2014).
49. Lachmann, A., et al. ChEA: transcription factor regulation inferred from integrating genome-wide ChIP-X experiments. Bioinformatics 26, 2438-2444 (2010).
50. Consortium, E. P. The ENCODE (ENCyclopedia Of DNA Elements) Project. Science 306, 636-640 (2004).
51. Rouillard, A. D., et al. The harmonizome: a collection of processed datasets gathered to serve and mine knowledge about genes and proteins. Database (Oxford) 2016(2016).
52. Davis, R. J. Signal transduction by the JNK group of MAP kinases. Cell 103, 239-252 (2000).
53. Sasaki, T., et al. Spatiotemporal regulation of c-Fos by ERK5 and the E3 ubiquitin ligase UBR1, and its biological role. Mol Cell 24, 63-75 (2006).
54. Ferrari, S., Bandi, H. R., Hofsteenge, J., Bussian, B. M. & Thomas, G. Mitogen-activated 70K S6 kinase. Identification of in vitro 40 S ribosomal S6 phosphorylation sites. J Biol Chem 266, 22770-22775 (1991).
55. Yamada, K., Saito, M., Matsuoka, H. & Inagaki, N. A real-time method of imaging glucose uptake in single, living mammalian cells. Nat Protoc 2, 753-762 (2007).
56. Kim, S. J., et al. A phase II study of everolimus (RAD001), an mTOR inhibitor plus CHOP for newly diagnosed peripheral T-cell lymphomas. Ann Oncol 27, 712-718 (2016).
57. Yukawa, M., et al. AP-1 activity induced by co-stimulation is required for chromatin opening during T cell activation. J Exp Med 217(2020).
58. Lynn, R. C., et al. c-Jun overexpression in CAR T cells induces exhaustion resistance. Nature 576, 293-300 (2019).
59. Qu, K., et al. Chromatin Accessibility Landscape of Cutaneous T Cell Lymphoma and Dynamic Response to HDAC Inhibitors. Cancer Cell 32, 27-41 e24 (2017).
60. Parekh, S., Ziegenhain, C., Vieth, B., Enard, W. & Hellmann, I. The impact of amplification on differential expression analyses by RNA-seq. Sci Rep 6, 25533 (2016).
61. Macosko, E. Z., et al. Highly Parallel Genome-wide Expression Profiling of Individual Cells Using Nanoliter Droplets. Cell 161, 1202-1214 (2015).
62. Love, M. I., Huber, W. & Anders, S. Moderated estimation of fold change and dispersion for RNA-seq data with DESeq2. Genome Biol 15, 550 (2014).
63. Mootha, V. K., et al. PGC-1alpha-responsive genes involved in oxidative phosphorylation are coordinately downregulated in human diabetes. Nat Genet 34, 267-273 (2003).
64. Subramanian, A., et al. Gene set enrichment analysis: a knowledge-based approach for interpreting genome-wide expression profiles. Proc Natl Acad Sci USA 102, 15545-15550 (2005).
65. Liberzon, A., et al. The Molecular Signatures Database (MSigDB) hallmark gene set collection. Cell Syst 1, 417-425 (2015).
66. Shechter, D., Dormann, H. L., Allis, C. D. & Hake, S. B. Extraction, purification and analysis of histones. Nat Protoc 2, 1445-1457 (2007).
67. Yuan, M., Breitkopf, S. B., Yang, X. & Asara, J. M. A positive/negative ion-switching, targeted mass spectrometry-based metabolomics platform for bodily fluids, cells, and fresh and fixed tissue. Nat Protoc 7, 872-881 (2012).
68. MacLean, B., et al. Skyline: an open source document editor for creating and analyzing targeted proteomics experiments. Bioinformatics 26, 966-968 (2010).
69. Lauterbach, M. A., et al. Toll-like Receptor Signaling Rewires Macrophage Metabolism and Promotes Histone Acetylation via ATP-Citrate Lyase. Immunity 51, 997-1011 e1017 (2019).
70. Cernilogar, F. M., et al. Pre-marked chromatin and transcription factor co-binding shape the pioneering activity of Foxa2. Nucleic Acids Res 47, 9069-9086 (2019).
71. Langmead, B. & Salzberg, S. L. Fast gapped-read alignment with Bowtie 2. Nat Methods 9, 357-359 (2012).
72. Li, H., et al. The Sequence Alignment/Map format and SAMtools. Bioinformatics 25, 2078-2079 (2009).
73. Tarasov, A., Vilella, A. J., Cuppen, E., Nijman, I. J. & Prins, P. Sambamba: fast processing of NGS alignment formats. Bioinformatics 31, 2032-2034 (2015).
74. Ramirez, F., Dundar, F., Diehl, S., Gruning, B. A. & Manke, T. deepTools: a flexible platform for exploring deep-sequencing data. Nucleic Acids Res 42, W187-191 (2014).
75. Feng, J., Liu, T., Qin, B., Zhang, Y. & Liu, X. S. Identifying ChIP-seq enrichment using MACS. Nat Protoc 7, 1728-1740 (2012).
76. Li, H. & Durbin, R. Fast and accurate short read alignment with Burrows-Wheeler transform. Bioinformatics 25, 1754-1760 (2009).
77. Choi, J., et al. Genomic landscape of cutaneous T cell lymphoma. Nat Genet 47, 1011-1019 (2015).
78. Daniels, J., et al. Cellular origins and genetic landscape of cutaneous gamma delta T cell lymphomas. Nat Commun 11, 1806 (2020).
79. Karczewski, K. J., et al. The mutational constraint spectrum quantified from variation in 141,456 humans. Nature 581, 434-443 (2020).
80. Bolotin, D. A., et al. MiXCR: software for comprehensive adaptive immunity profiling. Nat Methods 12, 380-381 (2015).
81. Corces, M. R., et al. An improved ATAC-seq protocol reduces background and enables interrogation of frozen tissues. Nat Methods 14, 959-962 (2017).
82. Bolger, A. M., Lohse, M. & Usadel, B. Trimmomatic: a flexible trimmer for Illumina sequence data. Bioinformatics 30, 2114-2120 (2014).

1. A method for treating a lymphoma in a subject in need thereof, comprising:
administering to the subject an effective amount of at least one PD-1 pathway agonist.

2. The method of claim 1, wherein the lymphoma is a T cell non-Hodgkin lymphoma.

3. The method of claim 1, wherein the PD-1 pathway agonist is selected from the group consisting of a PD-1 agonist, a PI3K-AKT-mTOR pathway inhibitor, a glycolysis inhibitor, an ACLY inhibitor, an AP1 inhibitor, or any combination of two or more thereof.

4. The method of claim 3, where the PD-1 agonist is selected from the group consisting of ImmTAA1 molecules, PD-L1, Rosnilimab, presolimab, PD-L1, PD-L2, or any combination of two or more thereof.

5. The method of claim 3, wherein the PI3K-AKT-mTOR pathway inhibitor is selected from the group consisting of a PI3K inhibitor, an AKT inhibitor, an mTOR inhibitor, or any combination of two or more thereof.

6. The method of claim 5, wherein the PI3K inhibitor is selected from the group consisting of alpelisib, AMG319, apitolisib, AZD8186, BKM120, BGT226, bimiralisib, buparlisib, CH5132799, copanlisib, CUDC-907, dactolisisb, duvelisib, GDC-0941, GDC-0084, gedatolisib, GSK2292767, GSK2636771, idelalisib, IPI-549, leniolisib, LY294002, LY3023414, nemiralisib, omipalisib, PF-04691502, pictilisib, pilaralisib, PX866, RV-1729, SAR260301, SAR245408, serabelisib, SF1126, sonolisib, taselisib, umbralisib, voxtalisib, VS-5584, wortmannin, WX-037, ZSTK474, or any combination of two or more thereof.

7. The method of claim 5, wherein the AKT inhibitor is selected from the group consisting of MK-2206, A-674563, A-443654, acetoxy-tirucallic acid, 3α- and 3β-acetoxy-tirucallic acids, afuresertib (GSK2110183), 4-amino-pyrido[2,3-d]pyrimidine derivative API-1, 3-aminopyrrolidine, anilinotriazole derivatives, ARQ751, ARQ 092, AT7867, AT13148, 7-azaindole, AZD5363, (−)-balanol derivatives, BAY 1125976, Boc-Phe-vinyl ketone, CCT128930, 3-chloroacetylindole, diethyl 6-methoxy-5,7-dihydroindolo [2,3-b]carbazole-2,10-dicarboxylate, diindolylmethane, 2,3-diphenylquinoxaline derivatives, DM-PIT-1, edelfosine, erucylphosphocholine, erufosine, frenolicin B, GSK-2141795, GSK690693, H-8, H-89, 4-hydroxynonenal, ilmofosine, imidazo-1,2-pyridine derivatives, indole-3-carbinol, ipatasertib, kalafungin, lactoquinomycin, medermycin, 3-methyl-xanthine, miltefosine, 1,6-naphthyridinone derivatives, NL-71-101, N-[(1-methyl-1H-pyrazol-4-yl)carbonyl]-N′-(3-bromophenyl)-thiourea, OSU-A9, perifosine, 3-oxo-tirucallic acid, PH-316, 3-phenyl-3H-imidazo[4,5-b]pyridine derivatives, 6-phenylpurine derivatives, PHT-427, PIT-1, PIT-2, 2-pyrimidyl-5-amidothiophene derivative, pyrrolo[2,3-d]pyrimidine derivatives, quinoline-4-carboxamide, 2-[4-(cyclohexa-1,3-dien-1-yl)-1H-pyrazol-3-yl]phenol, spiroindoline derivatives, triazolo[3,4-][1,6]naphthyridin-3(2H)-one derivative, triciribine, triciribine mono-phosphate active analogue, uprosertib, or any combination of two or more thereof.

8. The method of claim 5, wherein the mTOR inhibitor is selected from the group consisting of Torin, CCI-779, AZD2014, AZD8055, CC-223, dactolisib, everolimus, GSK2126458, Ku-0063794, Ku-0068650, MLN0128, OSI027, PP242, RapaLinks, rapamycin, ridaforolimus, sapanisertib, temsirolimus, vistusertib, WAY-600, WYE-687, WYE-354, XL765, or any combination of two or more thereof.

9. The method of claim 8, wherein the Torin is Torin 1 and/or Torin 2.

10. The method of claim 3, wherein the glycolysis inhibitor is selected from the group consisting of 2-deoxy-D-glucose, 3-bromopyruvic acid, 6-aminonicotinamide, lonidamine, oxythiamine chloride hydrochloride, or any combination of two or more thereof.

11. The method of claim 3, wherein the ACLY inhibitor is selected from the group consisting of BMS303141, SB204990, or any combination of two or more thereof.

12. The method of claim 3, wherein the AP1 inhibitor is selected from the group consisting of T-5224, SP100030, SPC-839, K1115A, Momordin I, or any combination of two or more thereof.

13. The method of claim 1, wherein the method comprises administering to the subject an additional therapeutic agent.

14. The method of claim 13, wherein the at least one PD-1 pathway agonist and the additional therapeutic agent are administered separately, sequentially, or simultaneously.

15. The method of claim 1, wherein the lymphoma is resistant to radiation therapy, chemotherapy or immunotherapy.

16. The method of claim 1, wherein the subject is non-responsive to at least one prior line of cancer therapy.

17. The method of claim 16, wherein the at least one prior line of cancer therapy is radiation therapy, chemotherapy or immunotherapy.

18. The method of claim 1, wherein the at least one PD-1 pathway agonist is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically.

19. The method of claim 1, wherein the subject is human.

20. The method of claim 1, wherein the subject exhibits decreased tumor growth, reduced tumor proliferation, lower tumor burden, or increased survival after administration of the at least one PD-1 pathway agonist.


SEQUENCE LISTING (25 sequences)

SEQ ID NO: 1 - DNA, 20 bp, synthetic construct
  source 1..20: mol_type = other DNA
gttcaatgagaaagttcttg

SEQ ID NO: 2 - DNA, 42 bp, Homo sapiens
  source 1..42: mol_type = genomic DNA
tgtgctgtatattcaggaggaggtgctgacggactcaccttt

SEQ ID NO: 3 - protein, 14 aa, Homo sapiens
CAVYSGGGADGLTF

SEQ ID NO: 4 - DNA, 42 bp, Homo sapiens; identical to SEQ ID NO: 2
  source 1..42: mol_type = genomic DNA

SEQ ID NO: 5 - protein, 14 aa, Homo sapiens
CAVYSGGGADGLTF

SEQ ID NO: 6 - DNA, 36 bp, Homo sapiens
  source 1..36: mol_type = genomic DNA
tgtgctctctacacgggcaggagagcacttactttt

SEQ ID NO: 7 - protein, 12 aa, Homo sapiens
CALYTGRRALTF

SEQ ID NO: 8 - DNA, 36 bp, Homo sapiens; identical to SEQ ID NO: 6
  source 1..36: mol_type = genomic DNA

SEQ ID NO: 9 - protein, 12 aa, Homo sapiens
CALYTGRRALTF

SEQ ID NO: 10 - DNA, 48 bp, Homo sapiens
  source 1..48: mol_type = genomic DNA
tgtgcagcaagcggcggatattacagcagtgcttccaagataatcttt

SEQ ID NO: 11 - protein, 16 aa, Homo sapiens
CAASGGYYSSASKIIF

SEQ ID NO: 12 - DNA, 48 bp, Homo sapiens; identical to SEQ ID NO: 10
  source 1..48: mol_type = genomic DNA

SEQ ID NO: 13 - protein, 16 aa, Homo sapiens
CAASGGYYSSASKIIF

SEQ ID NO: 14 - DNA, 45 bp, Homo sapiens
  source 1..45: mol_type = genomic DNA
tgtgccagcagtttcccccagggggctaactatggctacaccttc

SEQ ID NO: 15 - protein, 15 aa, Homo sapiens
CASSFPQGANYGYTF

SEQ ID NO: 16 - DNA, 45 bp, Homo sapiens; identical to SEQ ID NO: 14
  source 1..45: mol_type = genomic DNA

SEQ ID NO: 17 - protein, 15 aa, Homo sapiens
CASSFPQGANYGYTF

SEQ ID NO: 18 - DNA, 42 bp, Homo sapiens
  source 1..42: mol_type = genomic DNA
tgtgccagcagcgccttggggctcctctatggctacaccttc

SEQ ID NO: 19 - protein, 14 aa, Homo sapiens
CASSALGLLYGYTF

SEQ ID NO: 20 - DNA, 42 bp, Homo sapiens; identical to SEQ ID NO: 18
  source 1..42: mol_type = genomic DNA

SEQ ID NO: 21 - protein, 14 aa, Homo sapiens
CASSALGLLYGYTF

SEQ ID NO: 22 - DNA, 45 bp, Homo sapiens
  source 1..45: mol_type = genomic DNA
tgtgccagcagctctctagcgggagcgctcaatgagcagttcttc

SEQ ID NO: 23 - protein, 15 aa, Homo sapiens
CASSSLAGALNEQFF

SEQ ID NO: 24 - DNA, 45 bp, Homo sapiens; identical to SEQ ID NO: 22
  source 1..45: mol_type = genomic DNA

SEQ ID NO: 25 - protein, 15 aa, Homo sapiens
CASSSLAGALNEQFF